(19) United States
(12) Patent Application Publication
(10) Pub. No.: US 2024/0309113 A1
(43) Pub. Date: Sep. 19, 2024

(54) NOVEL ANTI-MASP-2 ANTIBODIES

(71) Applicant: SUZHOU TRANSCENTA THERAPEUTICS CO., LTD., Suzhou, Jiangsu (CN)

(72) Inventors: Xueming QIAN, Suzhou, Jiangsu (CN); Hongjun LI, Suzhou, Jiangsu (CN); Yi GU, Suzhou, Jiangsu (CN); Fei TENG, Suzhou, Jiangsu (CN); Di SUN, Suzhou, Jiangsu (CN)

(21) Appl. No.: 18/575,326

(22) PCT Filed: Jun. 30, 2022
(86) PCT No.: PCT/CN2022/102611

§ 371 Date: Dec. 29, 2023

(30) Foreign Application Priority Data
Jun. 30, 2021 (WO) PCT/CN2021/103715

(51) Int. Cl. C07K 16/40; A61K 47/68; A61K 49/00; A61K 51/10

(52) U.S. Cl. CPC C07K 16/40; A61K 47/6803; A61K 47/6871; A61K 49/0058; A61K 51/1075; C07K 2317/24; C07K 2317/31; C07K 2317/35; C07K 2317/92; C07K 2319/30

(57) ABSTRACT

Provided are anti-MASP-2 antibodies or antigen-binding fragments thereof, isolated polynucleotides encoding the same, pharmaceutical compositions comprising the same, and the uses thereof.

### FIELD OF THE INVENTION

The present disclosure generally relates to novel anti-MASP-2 antibodies and the uses thereof.

### BACKGROUND

The complement system comprises a complex array of enzymes and non-enzymatic proteins that is essential for the operation of the innate as well as the adaptive immune defense. There are 3 pathways to initiate complement activation: classical pathway, mannan-binding lectin (MBL) pathway and alternative pathway. These pathways depend on different molecules for their initiation, while they converge to generate the same set of effector molecules like membrane attack complex (MAC). All three pathways are important parts of innate immunity and play different roles in defending different infections. MBL is structurally related to the complement C1 subcomponent, C1q, and seems to activate the complement system through an associated serine protease known as MASP, which is similar to C1r and C1s of the classical pathway. MBL binds to specific carbohydrate structures found on the surface of a range of microorganisms, including bacteria, yeasts, parasitic protozoa and viruses, and exhibits antibacterial activity through killing mediated by the terminal, lytic complement components or by promoting phagocytosis (S Thiel et al., Nature, 386(6624):506-510 (1997), Noris M, et. al. 2013. JM.).

MASP-2 (MBL-associated serine protease 2) is involved in the complement system, which shows a striking homology with the MASP-1 and the two C1q-associated serine proteases C1r and C1s. Once the lectin recognizes and binds to the pathogen, the protozyme form of MASP-2 cleaves between CCP2 and SP domains (cleaved between the conserved R444 and I445), and turns into the active form consisting of two polypeptide chains (heavy chain/A chain and light chain/B chain) linked by disulfide bond (C434-C552) (A. B. W. Boldt et al., Human Immunology, 72(9): 753-760 (2011)). When MBL binds to a pathogen, MASP-2 is activated to cleave complement components C4 and C2 into C4a, C4b, C2a, and C2b, generating C3 convertase C4bC2b, subsequently C3 being converted to C3b by C4bC2b and finally form membrane attack complex (MAC) after C5 being converted to C5b by C3b. Activation of C3 finally leads to the formation of MAC, and then initiates a series of cascade activation processes of downstream complement system to stimulate innate immune response. Indeed C4b can activate the generation of C4d. C4d deposition could be a marker of complement activation. It has been shown that C4d-positive staining is an independent risk factor for the development of ESRD in IgAN (see, Clin J Am Soc Nephrol 9: 897-904, 2014) and kidney with C4d staining has significant short survival time than those without C4d staining in IgAN patients. C4D staining has also been detected in both kidneys of lupus nephritis and membrane glomerulonephritis patients. MASP-2 has been identified as a promising target for the treatment of autoimmune diseases.

In addition to its essential role in immune defense, the complement system contributes to tissue damage in many clinical conditions. Need remains for therapeutically effective complement inhibitors, such as novel MASP-2 antibodies, especially those with favorable high binding affinity and specificity, to prevent the adverse effects.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The complement system functions as an innate immunity that is capable of eliminating pathogenic microorganisms and its serine protease enzyme activate each other in a strictly ordered manner. There are many diseases associated with complement dysfunction, such as thrombotic microangiopathies (TMAs), atypical hemolytic uremic syndrome (aHUS), hematopoietic transplant-associated thrombotic microangiopathy (TA-TMA), lupus nephritis and IgA nephropathy. Most patients with these diseases were found uncontrolled complement activation and tissue injury by complement chronic attacking against endothelial cells. MASP-2 antibody that can block complement activation via the MBL pathway may be a potential therapeutic approach, which is also the mode of action of on-going clinical trials of OMS-721 from Omeros.

The present disclosure provides novel monoclonal anti-MASP-2 antibodies, amino acids and nucleotide sequences thereof, and uses thereof.

In one aspect, the present disclosure provides isolated antibodies or an antigen binding fragment thereof that specifically bind to MASP-2, wherein the antibody or antigen binding fragment thereof exhibits one or more of the following characteristics: a) having no cross-reactivity with mouse or rat; b) having longer serum half-life in monkey as compared with OMS721; c) having no cross-reactivity with C1s, C1r, MASP1 or MASP3; d) capable of selectively blocking the MBL pathway complement activation; e) capable of specifically binding to human MASP-2 at a K_{D} value of no more than 27.8 nM (or no more than 25 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, 0.01 nM, 0.009 nM, 0.008 nM, 0.007 nM, 0.006 nM, 0.005 nM, 0.004 nM, 0.003 nM, 0.002 nM, or 0.001 nM) as measured by Bio-Layer Interferometry; f) capable of blocking complement C3 activation at an IC50 of no more than 0.08 μg/mL (or no more than 0.07 μg/mL, 0.06 μg/mL, 0.05 μg/mL, 0.04 μg/mL, 0.03 μg/mL, 0.02 μg/mL, or 0.01 μg/mL) in 1% human serum as measured by ELISA assay, or at an IC50 of no more than 0.20 μg/mL (or no more than 0.15 μg/mL, 0.10 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) in 10% human serum as measured by ELISA assay; g) capable of blocking complement C3 activation in 50% human serum; h) capable of blocking complement C4 activation at an IC50 value of no more than 0.11 μg/mL (or no more than 0.10 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 2% human serum by ELISA assay, or at an IC50 value of no more than 0.69 μg/mL (or no more than 0.65 μg/mL, 0.6 μg/mL, 0.55 μg/mL, 0.5 μg/mL, 0.45 μg/mL, 0.4 μg/mL, 0.35 μg/mL, 0.3 μg/mL, 0.25 μg/mL, or 0.2 μg/mL, 0.15 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 10% human serum by ELISA assay; i) capable of blocking MAC formation at an IC50 value of no more than 0.27 μg/mL (or no more than 0.25 μg/mL, or 0.2 μg/mL, 0.15 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 2% human serum by ELISA assay.

In one aspect, the present disclosure provides isolated antibodies or antigen binding fragments thereof that specifically binds to MASP-2, comprising
a heavy chain CDR1 comprising the amino acid sequence of DYYIN (SEQ ID NO: 1), a heavy chain CDR2 comprising the amino acid sequence of WIFPGSX₁SX₂YX₃X₄X₅X₆FX₇X₈ (SEQ ID NO: 2), and
a heavy chain CDR3 comprising the amino acid sequence of GDRSGPFX₉Y (SEQ ID NO: 3); and/or
a light chain CDR1 comprising the amino acid sequence of KSSQSLLYSNGKTYLN (SEQ ID NO: 4),
a light chain CDR2 comprising the amino acid sequence of LVSKLDS (SEQ ID NO: 5), and
a light chain CDR3 comprising the amino acid sequence of VQX₁₀THFPFT (SEQ ID NO: 6);
wherein X1 is E, D or G, X₂ is A or P, X₃ is H or Y, X₄ is S or N, X₈ is E or Q, X₆ is K or N, X₇ is K or Q, X₈ is A or G, X₉ is A or P, and X₁₀ is V or G.

In certain embodiments, the antibodies or antigen binding fragments thereof provided herein comprise:
a heavy chain CDR1 comprising the amino acid sequence of SEQ ID NO: 1, and/or
a heavy chain CDR2 comprising the amino acid sequence selected from the group consisting of: SEQ ID NO: 7, SEQ ID NO: 8, SEQ ID NO: 9 and SEQ ID NO: 10, and/or
a heavy chain CDR3 comprising the amino acid sequence selected from the group consisting of: SEQ ID NO: 11 and SEQ ID NO: 12, and/or
a light chain CDR1 comprising the amino acid sequence of SEQ ID NO: 4, and/or
a light chain CDR2 comprising the amino acid sequence of SEQ ID NO: 5, and/or
a light chain CDR3 comprising the amino acid sequence selected from the group consisting of: SEQ ID NO: 13 and SEQ ID NO: 14.

In one aspect, the present disclosure provides isolated antibodies or antigen binding fragments thereof that specifically binds to MASP-2, comprising:
a heavy chain CDR1 comprising the amino acid sequence of SEQ ID NO: 1, a heavy chain CDR2 comprising the amino acid sequence of SEQ ID NO: 7, and a heavy chain CDR3 comprising the amino acid sequence of SEQ ID NO: 11; or
a heavy chain CDR1 comprising the amino acid sequence of SEQ ID NO: 1, a heavy chain CDR2 comprising the amino acid sequence of SEQ ID NO: 9, and a heavy chain CDR3 comprising the amino acid sequence of SEQ ID NO: 12; or
a heavy chain CDR1 comprising the amino acid sequence of SEQ ID NO: 1, a heavy chain CDR2 comprising the amino acid sequence of SEQ ID NO: 10, and a heavy chain CDR3 comprising the amino acid sequence of SEQ ID NO: 11; or
a heavy chain CDR1 comprising the amino acid sequence of SEQ ID NO: 1, a heavy chain CDR2 comprising the amino acid sequence of SEQ ID NO: 8, and a heavy chain CDR3 comprising the amino acid sequence of SEQ ID NO: 11.

In certain embodiments, the antibodies or antigen binding fragments thereof provided herein further comprise:
a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 14.

In certain embodiments, the antibodies or antigen binding fragments thereof provided herein comprise:
a heavy chain CDR1 comprising the amino acid sequence of SEQ ID NO: 1, a heavy chain CDR2 comprising the amino acid sequence of SEQ ID NO: 7, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
a heavy chain CDR1 comprising the amino acid sequence of SEQ ID NO: 1, a heavy chain CDR2 comprising the amino acid sequence of SEQ ID NO: 9, and a heavy chain CDR3 comprising the amino acid sequence of SEQ ID NO: 12, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
a heavy chain CDR1 comprising the amino acid sequence of SEQ ID NO: 1, a heavy chain CDR2 comprising the amino acid sequence of SEQ ID NO: 10, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 14; or
a heavy chain CDR1 comprising the amino acid sequence of SEQ ID NO: 1, a heavy chain CDR2 comprising the amino acid sequence of SEQ ID NO: 8, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13.

In certain embodiments, the antibodies or antigen binding fragments thereof provided herein comprise:
a heavy chain variable region comprising the amino acid sequence of SEQ ID NO: 15, or a sequence having at least 80% sequence identity thereof,
a heavy chain variable region comprising the amino acid sequence of SEQ ID NO: 17, or a sequence having at least 80% sequence identity thereof,
a heavy chain variable region comprising the amino acid sequence of SEQ ID NO: 18, or a sequence having at least 80% sequence identity thereof,
a heavy chain variable region comprising the amino acid sequence of SEQ ID NO: 20, or a sequence having at least 80% sequence identity thereof,
a heavy chain variable region comprising the amino acid sequence of SEQ ID NO: 22, or a sequence having at least 80% sequence identity thereof,
a heavy chain variable region comprising the amino acid sequence of SEQ ID NO: 24, or a sequence having at least 80% sequence identity thereof, or
a heavy chain variable region comprising the amino acid sequence of SEQ ID NO: 26, or a sequence having at least 80% sequence identity thereof.

In certain embodiments, the antibodies or antigen binding fragments thereof provided herein comprise:
a light chain variable region comprising the amino acid sequence of SEQ ID NO: 16, or a sequence having at least 80% sequence identity thereof,
a light chain variable region comprising the amino acid sequence of SEQ ID NO: 19, or a sequence having at least 80% sequence identity thereof,
a light chain variable region comprising the amino acid sequence of SEQ ID NO: 28, or a sequence having at least 80% sequence identity thereof, or
a light chain variable region comprising the amino acid sequence of SEQ ID NO: 30, or a sequence having at least 80% sequence identity thereof.

In certain embodiments, the antibodies or antigen binding fragments thereof provided herein comprise the heavy chain variable region comprising an amino acid sequence having at least 80% sequence identity to SEQ ID NO: 15, SEQ ID NO: 17, SEQ ID NO: 18, SEQ ID NO: 20, SEQ ID NO: 22, SEQ ID NO: 24, or SEQ ID NO: 26, and/or the light chain variable region comprises an amino acid sequence having at least 80% sequence identity to SEQ ID NO: 16, SEQ ID NO: 19, SEQ ID NO: 28, or SEQ ID NO: 30.

In certain embodiments, the antibodies or antigen binding fragments thereof provided herein comprise:
a heavy chain variable region comprising the amino acid sequence of SEQ ID NO: 15, and a light chain variable region comprising the amino acid sequence of SEQ ID NO: 16;
a heavy chain variable region comprising the amino acid sequence of SEQ ID NO: 17, and a light chain variable region comprising the amino acid sequence of SEQ ID NO: 16;
a heavy chain variable region comprising the amino acid sequence of SEQ ID NO: 18, and a light chain variable region comprising the amino acid sequence of SEQ ID NO: 19;
a heavy chain variable region comprising the amino acid sequence of SEQ ID NO: 20, and a light chain variable region comprising the amino acid sequence of SEQ ID NO: 28;
a heavy chain variable region comprising the amino acid sequence of SEQ ID NO: 20, and a light chain variable region comprising the amino acid sequence of SEQ ID NO: 30;
a heavy chain variable region comprising the amino acid sequence of SEQ ID NO: 22, and a light chain variable region comprising the amino acid sequence of SEQ ID NO: 28;
a heavy chain variable region comprising the amino acid sequence of SEQ ID NO: 22, and a light chain variable region comprising the amino acid sequence of SEQ ID NO: 30;
a heavy chain variable region comprising the amino acid sequence of SEQ ID NO: 24, and a light chain variable region comprising the amino acid sequence of SEQ ID NO: 28;
a heavy chain variable region comprising the amino acid sequence of SEQ ID NO: 24, and a light chain variable region comprising the amino acid sequence of SEQ ID NO: 30;
a heavy chain variable region comprising the amino acid sequence of SEQ ID NO: 26, and a light chain variable region comprising the amino acid sequence of SEQ ID NO: 28; or
a heavy chain variable region comprising the amino acid sequence of SEQ ID NO: 26, and a light chain variable region comprising the amino acid sequence of SEQ ID NO: 30.

In certain embodiments, the antibodies or antigen binding fragments thereof provided herein further comprise one or more amino acid residue mutations yet retain binding specificity to human MASP-2. In certain embodiments, at least one of the mutations is conservative substitution, or all of the mutations are conservative substitutions. In certain embodiments, at least one of the mutations is in one or more of the CDR sequences, and/or in one or more of the non-CDR sequences of the heavy chain variable region or light chain variable region.

In certain embodiments, the antibodies or antigen binding fragments thereof provided herein further comprise an immunoglobulin constant region, optionally comprise a heavy chain constant region of IgG, and/or a light chain constant region. In certain embodiments, the constant region comprises a mouse constant region, a rabbit constant region, or a human constant region, optionally the constant region comprises a constant region of human IgG1, IgG2, IgG3, or IgG4. In certain embodiments, the heavy chain constant region comprises one or more amino acid substitutions relative to a wild-type human IgG constant region at amino acid residue 252, 254 or 256, optionally the amino acid substitution at amino acid residue 252 is a substitution with tyrosine, the amino acid substitution at amino acid residue 254 is a substitution with threonine, an amino acid substitution at amino acid residue 256 is a substitution with glutamic acid. In certain embodiments, the heavy chain constant region comprises a sequence having at least 80% sequence identity thereof, provided that the amino acid residue 252 is substituted with tyrosine, the amino acid residue 254 is substituted with threonine, and the amino acid residue 256 is substituted with glutamic acid.

In certain embodiments, the antibodies or antigen binding fragments thereof provided herein are a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g. glycosylated antibody).

In certain embodiments, the antibodies or antigen binding fragments thereof provided herein are a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In certain embodiments, the antibodies or antigen binding fragments thereof provided herein specifically bind to MASP-2 and have no detectable cross-reactivity with C1s, C1r, MASP1 or MASP3.

In certain embodiments, the antibodies or antigen binding fragments thereof provided herein are linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, or a therapeutic agent.

In one aspect, the present disclosure further provides monoclonal antibodies or antigen binding fragments thereof, which compete for binding to MASP-2 with the antibodies or antigen binding fragments thereof provided herein.

In one aspect, the present disclosure further provides pharmaceutical compositions comprising the antibody or antigen binding fragment thereof provided herein and a pharmaceutically acceptable carrier.

In one aspect, the present disclosure further provides isolated polynucleotides encoding the antibodies or antigen binding fragments thereof provided herein.

In one aspect, the present disclosure further provides vectors comprising the isolated polynucleotides provided herein.

In one aspect, the present disclosure further provides host cells comprising the vectors provided herein.

In one aspect, the present disclosure further provides methods of expressing the antibodies or antigen binding fragments thereof provided herein, comprising culturing the host cells provided herein under the condition at which the polynucleotides provided herein are expressed.

In one aspect, the present disclosure further provides methods of inhibiting MASP-2 dependent complement activation in a subject in need thereof, comprising administering to the subject a therapeutically effective amount of the antibody or antigen binding fragment thereof provided herein or the pharmaceutical composition provided herein, thereby inhibiting MASP-2 dependent complement activation in the subject.

In one aspect, the present disclosure further provides methods of treating a disease or condition in a subject that would benefit from inhibition of MASP-2 dependent complement activation, comprising administering to the subject a therapeutically effective amount of the antibody or antigen binding fragment thereof provided herein or the pharmaceutical composition provided herein.

In one aspect, the present disclosure further provides methods of reducing level of serum C4 in a subject, comprising administering to the subject a therapeutically effective amount of the antibody or antigen binding fragment thereof provided herein or the pharmaceutical composition provided herein, thereby reducing the level of serum C4 in the subject.

In one aspect, the present disclosure further provides methods of treating a disease or condition in a subject that would benefit from reduction of serum C4 level, or treating or preventing a condition or a disease associated with abnormal serum C4 level, comprising administering to the subject a therapeutically effective amount of the antibody or antigen binding fragment thereof provided herein or the pharmaceutical composition provided herein.

In certain embodiments, said disease or condition is an autoimmune disease, a vascular condition, an ischemia-reperfusion injury, atherosclerosis, an inflammation, a pulmonary condition, extracorporeal reperfusion procedure, a musculoskeletal condition, a renal condition, a skin condition, an organ or tissue transplant procedure, a nervous system disorder or injury, a blood disorder, urogenital condition, a nonobese diabetes or a complication associated with Type 1 or Type 2 diabetes, cancer, endocrine disorder, or an ophthalmologic condition.

In certain embodiments, the autoimmune disease comprises thrombotic microangiopathies (TMAs), atypical hemolytic uremic syndrome (aHUS), hematopoietic transplant-associated thrombotic microangiopathy (TA-TMA), lupus nephritis, systemic lupus erythematosus (SLE) and IgA nephropathy, the vascular condition comprises a cardiovascular condition, a cerebrovascular condition, a peripheral (e.g., musculoskeletal) vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy and percutaneous transluminal coronary angioplasty (PTCA), the ischemia-reperfusion injury comprises an ischemia-reperfusion injury associated with aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures, the inflammation comprises inflammatory gastrointestinal disorder comprising pancreatitis, Crohn's disease, ulcerative colitis, irritable bowel syndrome and diverticulitis, the pulmonary condition comprises acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, emphysema, cystic fibrosis, SARS-CoV, MERS-CoV and SARS-CoV-2 (Covid-19) related condition, the extracorporeal reperfusion procedure comprises hemodialysis, plasmapheresis, leukapheresis, extracorporeal membrane oxygenator (ECMO), heparin-induced extracorporeal membrane oxygenation LDL precipitation (HELP) and cardiopulmonary bypass (CPB), the musculoskeletal condition comprises osteoarthritis, rheumatoid arthritis, juvenile rheumatoid arthritis, gout, neuropathic arthropathy, psoriatic arthritis, spondyloarthropathy, crystalline arthropathy and systemic lupus erythematosus (SLE), the renal condition comprises mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis and IgA nephropathy, the skin condition comprises psoriasis, autoimmune bullous dermatoses, eosinophilic spongiosis, bullous pemphigoid, Epidermolysis bullosa acquisita (EBA), herpes gestationis, thermal burn injury and chemical burn injury, the organ or tissue transplant procedure comprises organ allotransplantation, organ xenotransplantation organ and tissue graft, the nervous system disorder or injury comprises multiple sclerosis, myasthenia gravis, Huntington's disease, amyotrophic lateral sclerosis, Guillain Barre syndrome, reperfusion following stroke, degenerative discs, cerebral trauma, Parkinson's disease, Alzheimer's disease, Miller-Fisher syndrome, cerebral trauma and/or hemorrhage, demyelination and meningitis, the blood disorder comprises sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome, the urogenital condition comprises painful bladder disease, sensory bladder disease, chronic abacterial cystitis, interstitial cystitis, infertility, placental dysfunction and miscarriage and pre-eclampsia, the endocrine disorder comprises Hashimoto's thyroiditis, stress, anxiety and hormonal disorders involving regulated release of prolactin, growth or other insulin-like growth factor and adrenocorticotropin from the pituitary, the ophthalmologic condition comprises age-related macular degeneration.

In certain embodiments, the methods provided herein further comprise administration of a second therapeutic agent.

In certain embodiments, the subject is human. In certain embodiments, the administration is via oral, nasal, intravenous, subcutaneous, sublingual, or intramuscular administration.

In one aspect, the present disclosure further provides use of the antibodies or antigen binding fragments thereof provided herein in the manufacture of a medicament for treating a MASP-2 dependent complement activation related disease or condition in a subject.

In one aspect, the present disclosure further provides kits comprising the antibody or antigen binding fragment thereof provided herein.

### BRIEF DESCRIPTION OF FIGURES

FIG. 1 shows that MASP-2 antibodies block activation of the complement C3.

FIG. 2 shows that MASP-2 antibodies block activation of the complement C4.

FIG. 3 shows that MASP-2 antibodies block formation of the MAC.

FIG. 4 shows that chimeric and humanized MASP-2 antibodies block activation of the complement C3.

FIG. 5 shows that MASP-2 antibodies 129C10-hu and OMS721-analog block complement C4 activation in 2% human serum.

FIG. 6 shows that MASP-2 antibodies 129C10-hu and OMS721-analog block MAC formation in 2% human serum.

FIG. 7 shows that MASP-2 antibody blocks C3 activation in 1% human serum.

FIG. 8 shows that MASP-2 antibody blocks C3 activation in 10% human serum.

FIG. 9 shows that MASP-2 antibody blocks C3 activation in 50% human serum.

FIG. 10 shows that MASP-2 antibody blocks C4 activation in 10% human serum.

FIG. 11 shows affinity kinetics of MASP-2 antibody 129C10-hu to hMASP-2.

FIGS. 12A-12E show ELISA binding of MASP-2 antibody 129C10-Hu to human C1s (12A), C1r (12B), MASP1(12C), MASP3 (12D) and MASP2 (12E).

FIGS. 13A-13B show ELISA binding of MASP-2 antibodies 129C10-hu (13A) and OMS721-analog (13B) to human, cynomolgus, rat and mouse MASP2.

FIG. 14 shows cross reactivity of MASP-2 antibody 129C10-hu to cynomolgus MASP-2.

FIG. 15 shows selectivity of MASP-2 antibody 129C10-hu in neutralizing three complement activation pathways.

FIGS. 16A-16B show binding kinetic curves of MASP-2 antibodies 129C10-hu-WT (16A) and 129C10-hu-YTE (16B) to human FcRn.

FIG. 17 shows Pharmacokinetic (PK) results of MASP-2 antibodies 129C10-hu-WT and 129C10-hu-YTE in cynomolgus serum.

FIGS. 18A-18C show PK and Pharmacodynamic (PD) results of MASP-2 antibodies 129C10-hu-WT and 129C10-hu-YTE in cynomolgus serum.

FIG. 19 shows reduction effect of 129C10-hu on serum C4c in monkeys following the first dose.

FIG. 20 shows reduction effect of 129C10-hu on serum C4c in monkeys following the fourth dose.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (CH1, CH2, CH3, respectively); mammalian light chains are classified as λ or κ, while each light chain consists of a variable region (V_{L} for λ light chain or V_{K} for κ light chain, respectively) and a constant region (C_{L} for λ light chain or C_{K} for κ light chain, respectively). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5;186(3):651-63 (1985); Chothia, C. and Lesk, A. M., J. Mol. Biol., 196,901 (1987); Chothia, C. et al., Nature. December 21-28; 342 (6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of a, delta, epsilon, gamma, and heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (al heavy chain), or IgA2 (a2 heavy chain).

The term “bivalent” as used herein refers to an antibody or an antigen-binding fragment having two antigen-binding sites; the term “monovalent” refers to an antibody or an antigen-binding fragment having only one single antigen-binding site; and the term “multivalent” refers to an antibody or an antigen-binding fragment having multiple antigen-binding sites. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “antigen-binding fragment” as used herein refers to an antibody fragment formed from a portion of an antibody comprising 1, 2, or 3 CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′. “Fv” with regard to an antibody refers to the smallest fragment of the antibody to bear the complete antigen-binding site. An Fv fragment consists of the variable region of a single light chain bound to the variable region of a single heavy chain.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), and complement dependent cytotoxicity (CDC), but does not function in antigen binding.

“Single-chain Fv-Fc antibody” or “scFv-Fc” refers to an engineered antibody consisting of a scFv connected to the Fc region of an antibody.

“Camelized single domain antibody”, “heavy chain antibody”, or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes). In certain embodiments, an “scFv dimer” is a bivalent diabody or bivalent ScFv (BsFv) comprising V_{H}—V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rat. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, constant regions derived from human.

As used herein, the term “MASP-2” refers to the mannan-binding lectin associated serine protease 2, which is a crucial member of the MBL pathway of the complement system. The human, mouse and cynomolgus MASP-2 amino acid and nucleic acid sequences can be found in a public database, such as GenBank, UniProt and Swiss-Prot. As used herein, the term MASP-2 includes proteins comprising mutations, e.g., point mutations, fragments, insertions, deletions and splice variants of full length wild-type MASP-2. In certain embodiments, the human MASP-2 protein comprises an amino acid sequence of SEQ ID NO: 39. In certain embodiments, the mouse MASP-2 protein comprises an amino acid sequence of SEQ ID NO: 40. In certain embodiments, the cynomolgus MASP-2 protein comprises an amino acid sequence of SEQ ID NO: 43. In certain embodiments, a chimeric MASP-2 protein comprising mouse MASP-2 CUB1-EGF-CUB2 domain (residues 20-297) and human MASP-2 CCP1-CCP2-SP domain (residues 298-686) were synthesized and has an amino acid sequence of SEQ ID NO: 42.

The term “specific binding” or “specifically binds” as used herein refers to a non-random binding reaction between two molecules, such as for example between an antibody and an antigen. In certain embodiments, the antibodies or antigen-binding fragments provided herein specifically bind to human MASP-2 with a binding affinity (K_{D}) of ≤10⁻⁶ M (e.g., ≤5×10⁻⁸ M, ≤2×10⁻⁸ M, ≤10⁻⁸ M, 5×10⁻⁹ M, ≤2×10⁻⁹ M, ≤10⁻⁹ M, ≤5×10⁻¹⁰ M, ≤2×10⁻¹⁰ M, ≤10⁻¹⁰ M, ≤5×10⁻¹¹ M, ≤2×10⁻¹¹ M, 10⁻¹¹ M, ≤5×10⁻¹² M, ≤4×10⁻¹² M, ≤3×10⁻¹² M, ≤2×10⁻¹² M, or ≤10⁻¹² M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method, Bio-Layer Interferometry and flow cytometry (such as FACS) method. In certain embodiments, the K_{D} value can be appropriately determined by using ForteBio method.

The ability to “block binding” or “compete for the same epitope” as used herein refers to the ability of an antibody or antigen-binding fragment to inhibit the binding interaction between two molecules (e.g., MASP-2 protein and an anti-MASP-2 antibody) to any detectable degree. In certain embodiments, an antibody or antigen-binding fragment that blocks binding between two molecules inhibits the binding interaction between the two molecules by at least 50%, at least 60%, at least 70%, at least 75%, at least 80%, at least 85%, or at least 90%. In certain embodiments, this inhibition may be greater than 60%, greater than 70%, greater than 75%, greater than 80%, greater than 85%, or greater than 90%.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen (e.g., human/monkey MASP-2) by at least 50%, at least 60%, at least 70%, at least 75%, at least 80%, at least 85%, or at least 90%, then the antibody or antigen-binding fragment may be considered to bind the same/closely related epitope as the reference antibody.

Those skilled in the art will recognize that it is possible to determine, without undue experimentation, if a human monoclonal antibody binds to the same epitope as the antibody of present disclosure by ascertaining whether the former prevents the latter from binding to a MASP-2 antigen polypeptide. If the test antibody competes with the antibody of present disclosure, as shown by a decrease in binding by the antibody of present disclosure to the MASP-2 antigen polypeptide, then the two antibodies bind to the same, or a closely related, epitope. Or if the binding of a test antibody to the MASP-2 antigen polypeptide was inhibited by the antibody of present disclosure, then the two antibodies bind to the same, or a closely related, epitope.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector has been introduced.

The complement system has been implicated in the pathogenesis of numerous acute and chronic diseases or conditions, including: myocardial infarction, stroke, acute respiratory distress syndrome (ARDS), reperfusion injury, septic shock, capillary leakage following thermal burns, post cardiopulmonary bypass inflammation, transplant rejection, rheumatoid arthritis, multiple sclerosis, myasthenia gravis, and Alzheimer's disease. In almost all of these diseases or conditions, complement is not the cause but is one of several factors involved in pathogenesis. Complement activation may be a major pathological mechanism and represents an effective point for clinical control in many of these disease states. Participating the MBL pathway, one of the three major complement activation pathways, MASP-2 may also be involved in the pathogenesis of many diseases or conditions. In some embodiments, the disease or condition that is related to MASP-2 dependent complement activation is an autoimmune disease, a vascular condition, an ischemia-reperfusion injury, atherosclerosis, an inflammation, a pulmonary condition, extracorporeal reperfusion procedure, a musculoskeletal condition, a renal condition, a skin condition, an organ or tissue transplant procedure, a nervous system disorder or injury, a blood disorder, urogenital condition, a nonobese diabetes or a complication associated with Type 1 or Type 2 diabetes, cancer, endocrine disorder, or an ophthalmologic condition.

The term “mannan-binding lectin” (“MBL”) is equivalent to mannan-binding protein (“MBP”). The term “membrane attack complex” (“MAC”, also referred to as C5b-9) refers to a complex of the terminal five complement components (C5-C9) that inserts into and disrupts cell membranes.

An “autoimmune disease” as used herein refers to a pathophysiological state wherein immune responses are directed against, and damage, the body's own tissues (autoimmunity). Examples of autoimmune disease include but are not limited to thrombotic microangiopathies (TMAs), atypical hemolytic uremic syndrome (aHUS), hematopoietic transplant-associated thrombotic microangiopathy (TA-TMA), lupus nephritis, systemic lupus erythematosus (SLE) and IgA nephropathy.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

### Anti-MASP-2 Antibody

The present disclosure provides anti-MASP-2 antibodies and antigen-binding fragments thereof comprising one or more (e.g., 1, 2, 3, 4, 5, or 6) CDR sequences of an anti-MASP-2 antibody selected from the group consisting of 129C10 mouse/chimeric, 160D10 mouse/chimeric, 125D5 mouse/chimeric, 129C10 HaLa, 129C10 HaLb, 129C10 HbLa, 129C10 HbLb, 129C10 HcLa, 129C10 HcLb, 129C10 HdLa, or 129C10 HdLb.

“129C10 mouse/chimeric” as used herein refers to a mouse monoclonal antibody having a heavy chain variable region of SEQ ID NO: 15, and a light chain variable region of SEQ ID NO: 16.

“160D10 mouse/chimeric” as used herein refers to a mouse monoclonal antibody having a heavy chain variable region of SEQ ID NO: 17, and a light chain variable region of SEQ ID NO: 16.

“125D5 mouse/chimeric” as used herein refers to a mouse monoclonal antibody having a heavy chain variable region of SEQ ID NO: 18, and a light chain variable region of SEQ ID NO: 19.

“129C10 HaLa” or “129C10-hu” as used herein refers to a humanized antibody based on 129C10 mouse/chimeric that comprises a heavy chain variable region of SEQ ID NO: 20, and a light chain variable region of SEQ ID NO: 28.

“129C10 HaLb” as used herein refers to a humanized antibody based on 129C10 mouse/chimeric that comprises a heavy chain variable region of SEQ ID NO 20, and a light chain variable region of SEQ ID NO: 30.

“129C10 HbLa” as used herein refers to a humanized antibody based on 129C10 mouse/chimeric that comprises a heavy chain variable region of SEQ ID NO: 22, and a light chain variable region of SEQ ID NO: 28.

“129C10HbLb” as used herein refers to a humanized antibody based on 129C10 mouse/chimeric that comprises a heavy chain variable region of SEQ ID NO: 22, and a light chain variable region of SEQ ID NO: 30.

“129C10 HcLa” as used herein refers to a humanized antibody based on 129C10 mouse/chimeric that comprises a heavy chain variable region of SEQ ID NO: 24, and a light chain variable region of SEQ ID NO: 28.

“129C10 HcLb” as used herein refers to a humanized antibody based on 129C10 mouse/chimeric that comprises a heavy chain variable region of SEQ ID NO: 24, and a light chain variable region of SEQ ID NO: 30.

“129C10 HdLa” as used herein refers to a humanized antibody based on 129C10 mouse/chimeric that comprises a heavy chain variable region of SEQ ID NO: 26, and a light chain variable region of SEQ ID NO: 28.

“129C10 HdLb” as used herein refers to a humanized antibody based on 129C10 mouse/chimeric that comprises a heavy chain variable region of SEQ ID NO: 26, and a light chain variable region of SEQ ID NO: 30.

Antibodies 129C10 HaLa, 129C10 HaLb, 129C10 HbLa, and 129C10 HbLb share the same CDRs as the 129C10 mouse/chimeric antibody.

Antibodies 129C10 HcLa, 129C10 HcLb, 129C10 HdLa, and 129C10 HdLb share the same CDRs.

Table 1 shows the CDR sequences of the three mouse anti-MASP-2 antibodies, and of the eight humanized antibodies.

TABLE 1
CDR sequences of mouse anti-MASP-2 antibodies and humanized 129C10
antibodies

| | | CDR1 | CDR2 | CDR3 |
|---|---|---|---|---|
| 129C10 | VH | SEQ ID NO: 1 | SEQ ID NO: 7 | SEQ ID NO: 11 |
| mouse/chimeric | | DYYIN | WIFPGSESAYHSEK | GDRSGPFAY |
| antibody, and | | | FKA | |
| 129C10 | | | | |
| HaLa/HaLb/ | | | | |
| HbLa/HbLb | | | | |
| 129C10 | VL | SEQ ID NO: 4 | SEQ ID NO: 5 | SEQ ID NO: 13 |
| mouse/chimeric | | KSSQSLLYSNGK | LVSKLDS | VQVTHFPFT |
| antibody, and | | TYLN | | |
| 129C10 | | | | |
| HaLa/HaLb/ | | | | |
| HbLa/HbLb | | | | |
| 160D10 | VH | SEQ ID NO: 1 | SEQ ID NO: 9 | SEQ ID NO: 12 |
| mouse/chimeric | | DYYIN | WIFPGSDSAYYNE | GDRSGPFPY |
| antibody | | | KFKG | |
| 160D10 | VL | SEQ ID NO: 4 | SEQ ID NO: 5 | SEQ ID NO: 13 |
| mouse/chimeric | | KSSQSLLYSNGK | LVSKLDS | VQVTHFPFT |
| antibody | | TYLN | | |
| 125D5 | VH | SEQ ID NO: 1 | SEQ ID NO: 10 | SEQ ID NO: 11 |
| mouse/chimeric | | DYYIN | WIFPGSGSPYYNE | GDRSGPFAY |
| antibody | | | NFKG | |
| 125D5 | VL | SEQ ID NO: 4 | SEQ ID NO: 5 | SEQ ID NO: 14 |
| mouse/chimeric | | KSSQSLLYSNGK | LVSKLDS | VQGTHFPFT |
| antibody | | TYLN | | |
| 129C10 | VH | SEQ ID NO: 1 | SEQ ID NO: 8 | SEQ ID NO: 11 |
| HcLa/HcLb/ | | DYYIN | WIFPGSESAYHSQ | GDRSGPFAY |
| HdLa/HdLb | | | KFQG | |
| antibody | | | | |
| 129C10 | VL | SEQ ID NO: 4 | SEQ ID NO: 5 | SEQ ID NO: 13 |
| HcLa/HcLb/ | | KSSQSLLYSNGK | LVSKLDS | VQVTHFPFT |
| HdLa/HdLb | | TYLN | | |
| antibody | | | | |

Table 2 shows the sequences of heavy chain and light chain variable regions of the three mouse anti-MASP-2 antibodies (Table 2-1), of heavy chain and light chain variable regions of the humanized 129C10 antibodies (Table 2-2), and of the 129C10-hu-YTE antibody (Table 2-3).

TABLE 2
Sequences of anti-MASP-2 antibodies
2-1 Variable region sequences of murine or chimeric antibodies

| | | |
|---|---|---|
| 129C10 | HC | Amino acid sequence (SEQ ID NO: 15): |
| mouse/chimeric | | QVQLQQSGPELEKPGTSVKISCEASGYTFTDYYINWVQQR |
| antibody | | PGQGLEWIGWIFPGSESAYHSEKFKAKATLTVDTSSTTAY |
| | | MLLTSLTSEDSAVYFCTRGDRSGPFAYWGQGTLVTVSA |
| 129C10 | LC | Amino acid sequence (SEQ ID NO: 16): |
| mouse/chimeric | | DVVMTQTPLTLSVTIGQPASISCKSSQSLLYSNGKTYLNWL |
| antibody | | LQRPGQSPKRLIYLVSKLDSGVPDRFTGSGSGADFTLKISR |
| | | VEAEDLGVYYCVQVTHFPFTFGTGTKLEIK |
| 160D10 | HC | Amino acid sequence (SEQ ID NO: 17): |
| mouse/chimeric | | QVQLQQSGPELVKPGASVKISCKASGYTFTDYYINWVKQ |
| antibody | | RPGQGLEWVGWIFPGSDSAYYNEKFKGKATLTVDTSSST |
| | | AYMLLSSLTSEDSAVYFCARGDRSGPFPYWGQGTLVTVS |
| | | A |
| 160D10 | LC | Amino acid sequence (SEQ ID NO: 16): |
| mouse/chimeric | | DVVMTQTPLTLSVTIGQPASISCKSSQSLLYSNGKTYLNWL |
| antibody | | LQRPGQSPKRLIYLVSKLDSGVPDRFTGSGSGADFTLKISR |
| | | VEAEDLGVYYCVQVTHFPFTFGTGTKLEIK |
| 125D5 | HC | Amino acid sequence (SEQ ID NO: 18): |
| mouse/chimeric | | QVQLQQSGPDLVKPGTSVKISCTASGYTFTDYYINWVKQR |
| antibody | | PGQGLEWIGWIFPGSGSPYYNENFKGRAMFTVDYSSSSAY |
| | | MLLSSLTSEDSAVYFCTRGDRSGPFAYWGQGTLVTVSA |
| 125D5 | LC | Amino acid sequence (SEQ ID NO: 19): |
| mouse/chimeric | | DVVMTQTPLTLSVTIGQPASISCKSSQSLLYSNGKTYLNWL |
| antibody | | LQRPGQSPKRLIYLVSKLDSGVPDRFTGSGSGADFTLKISR |
| | | VEAEDLGVYYCVQGTHFPFTFGTGTKLEIK |

2-2 Variable region sequences of humanized 129C10

| | | |
|---|---|---|
| Heavy chain | Ha | Amino acid sequence (SEQ ID NO: 20): |
| variable | | QVQLVQSGAEVKKPGASVKVSCKASGYTFTDYYINWVRQ |
| region of | | APGQGLEWMGWIFPGSESAYHSEKFKARVTMTVDTSISTA |
| 129C10 | | YMELSRLRSDDTAVYYCTRGDRSGPFAYWGQGTLVTVSS |
| Heavy chain | Hb | Amino acid sequence (SEQ ID NO: 22): |
| variable | | QVQLVQSGAEVKKPGASVKVSCKASGYTFTDYYINWVRQ |
| region of | | APGQGLEWMGWIFPGSESAYHSEKFKARATLTVDTSISTA |
| humanized | | YMELSRLRSDDTAVYYCTRGDRSGPFAYWGQGTLVTVSS |
| 129C10 | | |
| Heavy chain | Hc | Amino acid sequence (SEQ ID NO: 24): |
| variable | | QVQLVQSGAEVKKPGASVKVSCKASGYTFTDYYINWVRQ |
| region of | | APGQGLEWMGWIFPGSESAYHSQKFQGRVTMTVDTSIST |
| humanized | | AYMELSRLRSDDTAVYYCTRGDRSGPFAYWGQGTLVTVS |
| 129C10 | | S |
| Heavy chain | Hd | Amino acid sequence (SEQ ID NO: 26): |
| variable | | QVQLVQSGAEVKKPGASVKVSCKASGYTFTDYYINWVRQ |
| region of | | APGQGLEWMGWIFPGSESAYHSQKFQGRATLTVDTSISTA |
| humanized | | YMELSRLRSDDTAVYYCTRGDRSGPFAYWGQGTLVTVSS |
| 129C10 | | |
| Light chain | La | Amino acid sequence (SEQ ID NO: 28): |
| variable | | DVVMTQSPLSLPVTLGQPASISCKSSQSLLYSNGKTYLNW |
| region of | | LQQRPGQSPRRLIYLVSKLDSGVPDRFSGSGSGTDFTLKIS |
| humanized | | RVEAEDVGVYYCVQVTHFPFTFGQGTKLEIK |
| 129C10 | | |
| Light chain | Lb | Amino acid sequence (SEQ ID NO: 30): |
| variable | | DVVMTQSPLSLPVTLGQPASISCKSSQSLLYSNGKTYLNW |
| region of | | LQQRPGQSPRRLIYLVSKLDSGVPDRFSGSGSGADFTLKIS |
| humanized | | RVEAEDVGVYYCVQVTHFPFTFGQGTKLEIK |
| 129C10 | | |

2-3 Sequences of 129C10-hu-YTE antibody

| | | |
|---|---|---|
| Heavy chain | | Amino acid sequence (SEQ ID NO: 35): |
| constant | | ASTKGPSVFPLAPCSRSTSESTAALGCLVKDYFPEPVTVSW |
| region of | | NSGALTSGVHTFPAVLQSSGLYSLSSVVTVPSSSLGTKTYT |
| IgG4 YTE | | CNVDHKPSNTKVDKRVESKYGPPCPPCPAPEFLGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSL |
| | | SLSLGK |
| Light chain | | Amino acid sequence (SEQ ID NO: 34): |
| constant | | RTVAAPSVFIFPPSDEQLKSGTASVVCLLNNFYPREAKVQ |
| region of | | WKVDNALQSGNSQESVTEQDSKDSTYSLSSTLTLSKADYE |
| IgG4 | | KHKVYACEVTHQGLSSPVTKSFNRGEC |
| Heavy chain | HC | Amino acid sequence (SEQ ID NO: 36): |
| of | | QVQLVQSGAEVKKPGASVKVSCKASGYTFTDYYINWVRQ |
| Hu129C10 | | APGQGLEWMGWIFPGSESAYHSEKFKARVTMTVDTSISTA |
| (hIgG4_YTE | | YMELSRLRSDDTAVYYCTRGDRSGPFAYWGQGTLVTVSS |
| kappa) | | ASTKGPSVFPLAPCSRSTSESTAALGCLVKDYFPEPVTVSW |
| antibody | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSL |
| | | SLSLGK |
| Light chain | LC | Amino acid sequence (SEQ ID NO: 29): |
| of | | DVVMTQSPLSLPVTLGQPASISCKSSQSLLYSNGKTYLNW |
| Hu129C10 | | LQQRPGQSPRRLIYLVSKLDSGVPDRFSGSGSGTDFTLKIS |
| (hIgG4_YTE | | RVEAEDVGVYYCVQVTHFPFTFGQGTKLEIKRTVAAPSVF |
| kappa) | | IFPPSDEQLKSGTASVVCLLNNFYPREAKVQWKVDNALQS |
| antibody | | GNSQESVTEQDSKDSTYSLSSTLTLSKADYEKHKVYACEV |
| | | THQGLSSPVTKSFNRGEC |

CDRs are known to be responsible for antigen binding, however, it has been found that not all of the 6 CDRs are indispensable or unchangeable. In other words, it is possible to replace or change or modify 1, 2, or 3 CDRs in anti-MASP-2 antibodies 129C10 mouse/chimeric, 160D10 mouse/chimeric, 125D5 mouse/chimeric, 129C10 HaLa, 129C10 HaLb, 129C10 HbLa, 129C10 HbLb, 129C10 HcLa, 129C10 HcLb, 129C10 HdLa, or 129C10 HdLb antibodies, yet substantially retain the specific binding affinity to MASP-2.

In certain embodiments, the anti-MASP-2 antibodies and the antigen-binding fragments provided herein comprise a heavy chain CDR3 sequence of one of the anti-MASP-2 antibodies 129C10 mouse/chimeric, 160D10 mouse/chimeric, 125D5 mouse/chimeric, 129C10 HaLa, 129C10 HaLb, 129C10 HbLa, 129C10 HbLb, 129C10 HcLa, 129C10 HcLb, 129C10 HdLa, or 129C10 HdLb antibodies. In certain embodiments, the anti-MASP-2 antibodies and the antigen-binding fragments provided herein comprise a heavy chain CDR3 sequence selected from the group consisting of SEQ ID NOs: 3, 11, and 12. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the antibodies and antigen-binding fragments thereof provided herein comprise suitable framework region (FR) sequences, as long as the antibodies and antigen-binding fragments thereof can specifically bind to MASP-2. The CDR sequences provided in Table 1 can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment comprise human heavy/light chain FR1-4.

The exemplary humanized anti-MASP-2 antibodies, 129C10 HaLa, 129C10 HaLb, 129C10 HbLa, 129C10 HbLb, 129C10 HcLa, 129C10 HcLb, 129C10 HdLa, and 129C10 HdLb antibodies all retained the specific binding affinity to MASP-2, and are at least comparable to, or even better than, the parent mouse antibodies in that aspect.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure to reduce or avoid immunogenicity and/or improve or retain the binding activity or binding affinity.

In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. In certain embodiments, the one or more amino acid residues are mutated, for example, back-mutated to the corresponding residue found in the non-human parent antibody (e.g. in the mouse framework region) from which the CDR sequences are derived. Suitable positions for mutations can be selected by a skilled person following principles known in the art. For example, a position for mutation can be selected where: 1) the residue in the framework of the human germline sequence is rare (e.g. in less than 20% or less than 10% in human variable region sequence); 2) the position is immediately adjacent to one or more of the 3 CDR's in the primary sequence of the human germline chain, as it is likely to interact with residues in the CDRs; or 3) the position is close to CDRs in a 3-dimensional model, and therefore can have a good probability of interacting with amino acids in the CDR. The residue at the selected position can be mutated back to the corresponding residue in the parent antibody, or to a residue which is neither the corresponding residue in human germline sequence nor in parent antibody, but to a residue typical of human sequences, i.e. that occurs more frequently at that position in the known human sequences belonging to the same subgroup as the human germline sequence (see U.S. Pat. No. 5,693,762).

In certain embodiments, the humanized heavy and light chains of the antibodies and antigen-binding fragments thereof provided herein are substantially non-immunogenic in humans and retain substantially the same affinity as or even higher affinity than the parent antibody to MASP-2.

In certain embodiments, the humanized antibodies and antigen-binding fragment thereof provided herein comprise one or more heavy chain FR sequences of human germline framework sequence VH/1-2, and/or one or more light chain FR sequences of human germline framework sequence VK/2-30 without or without back mutations. Back mutations can be introducted into the human germline framework sequence, if needed. In certain embodiments, the humanized antibody 129C10 may contain one or more mutations selected from the group consisting of: R71V, A93T, V67A, M69L, all based on Kabat numbering, in heavy chain framework sequence VH/1-2, and/or A65G, K64Q, E61Q, all based on Kabat numbering, in heavy chain CDR2. The humanized antibody 129C10 may contain one or more back mutations selected from the group consisting of: F36L and T69A, all based on Kabat numbering, in light chain framework sequence VK/2-30.

In certain embodiments, the antibodies and antigen-binding fragments thereof provided herein comprise a heavy chain variable domain sequence selected from the group consisting of: SEQ ID NO: 15, SEQ ID NO: 17, SEQ ID NO: 18, SEQ ID NO: 20, SEQ ID NO: 22, SEQ ID NO: 24, and SEQ ID NO: 26. In certain embodiments, the antibodies and antigen-binding fragments thereof provided herein comprise a light chain variable domain sequence selected from the group consisting of: SEQ ID NO: 16, SEQ ID NO: 19, SEQ ID NO: 28, and SEQ ID NO: 30.

In some embodiments, the anti-MASP-2 antibodies and the antigen-binding fragments provided herein comprise all or a portion of the heavy chain variable domain and/or all or a portion of the light chain variable domain. In one embodiment, the anti-MASP-2 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-MASP-2 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In some embodiments, the anti-MASP-2 antibodies and antigen-binding fragments thereof provided herein have a constant region of an immunoglobulin (Ig), optionally a human Ig, optionally a human IgG. In some embodiments, the anti-MASP-2 antibodies and antigen-binding fragments thereof provided herein comprises a constant region of human IgG1, IgG2, IgG3, or IgG4.

In certain embodiments, the anti-MASP-2 antibodies and antigen-binding fragments thereof provided herein comprises a constant region of IgG4 or IgG2 isotype, which has reduced or depleted effector function. Binding affinity of the antibody and antigen-binding fragment provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, Bio-Layer Interferometry.

In certain embodiments, the anti-MASP-2 antibodies and antigen-binding fragments thereof provided herein are capable of specifically binding to human/Cynomolgus monkey MASP-2. For example, human/Cynomolgus monkey MASP-2 DNA sequence can be cloned into an expression vector, and then transfected and expressed in CHO cells such that human/Cynomolgus monkey MASP-2 protein can be expressed on the surface of the transfected CHO cells.

In some embodiments, the anti-MASP-2 antibodies and the antigen-binding fragments thereof provided herein are capable of specifically binding to human MASP-2 expressed on surface of cells with a binding affinity (K_{D}) of no more than 3×10⁻⁸M, no more than 1×10⁻⁸M, no more than 9×10⁻⁹M, no more than 8×10⁻⁹M, no more than 7×10⁻⁹M, no more than 6×10⁻⁹M, no more than 5×10⁻⁹M, no more than 4×10⁻⁹M, no more than 3×10⁻⁹M, no more than 2×10⁻⁹M, no more than 1×10⁻⁹M, no more than 9×10⁻¹⁰ M, no more than 8×10⁻¹⁰ M, no more than 7×10⁻¹⁰ M, no more than 6×10⁻¹⁰ M, no more than 5×10⁻¹⁰ M, no more than 4×10⁻¹⁰ M, no more than 3×10⁻¹⁰M, no more than 2×10⁻¹⁰ M, no more than 1×10⁻¹⁰ M, no more than 5×10⁻¹¹M, no more than 1×10⁻¹¹M, no more than 5×10⁻¹²M, no more than 1×10⁻¹²M as measured by Bio-Layer Interferometry.

Binding of the antibodies to MASP-2 can be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assay. In certain embodiments, the antibodies and the fragments thereof provided herein specifically bind to human MASP-2 expressed on a cell with an EC50 of no more than 0.02 μg/mL, no more than 0.015 μg/mL, no more than 0.01 μg/mL, no more than 0.005 μg/mL, no more than 0.001 μg/mL, by ELISA assay.

Binding of the antibodies to MASP-2 can also be represented by “half maximal inhibitory concentration” (IC50), which measures the potency of a substance in inhibiting a specific biological or biochemical function. IC50 is a quantitative measure that indicates how much of a particular inhibitory substance (e.g. drug) is needed to inhibit, in vitro, a given biological process or biological component by 50%. The IC50 value can be measured by methods known in the art, for example, sandwich assay such as ELISA, Western Blot, flow cytometry assay, and other binding assay.

In certain embodiments, the antibodies and the fragments thereof provided herein are capable of blocking complement C3 activation at an IC50 of no more than 0.08 μg/mL (or no more than 0.07 μg/mL, 0.06 μg/mL, 0.05 μg/mL, 0.04 μg/mL, 0.03 μg/mL, 0.02 μg/mL, or 0.01 μg/mL) in 1% human serum, or at an IC50 of no more than 0.20 μg/mL (or no more than 0.15 μg/mL, 0.10 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) in 10% human serum. In certain embodiments, the antibodies and the fragments thereof provided herein are capable of blocking complement C3 activation in 50% human serum.

In certain embodiments, the antibodies and the fragments thereof provided herein are capable of blocking complement C4 activation at an IC50 value of no more than 0.11 μg/mL (or no more than 0.10 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 2% human serum, or at an IC50 value of no more than 0.69 μg/mL (or no more than 0.65 μg/mL, 0.6 μg/mL, 0.55 μg/mL, 0.5 μg/mL, 0.45 μg/mL, 0.4 μg/mL, 0.35 μg/mL, 0.3 μg/mL, 0.25 μg/mL, or 0.2 μg/mL, 0.15 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 10% human serum.

In certain embodiments, the antibodies and the fragments thereof provided herein are capable of blocking MAC formation at an IC50 value of no more than 0.27 μg/mL (or no more than 0.25 μg/mL, or 0.2 μg/mL, 0.15 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, or 0.01 μg/mL) as measured in 2% human serum by ELISA.

In certain embodiments, the anti-MASP-2 antibodies and antigen-binding fragments thereof provided herein binds to Cynomolgus monkey MASP-2. In certain embodiments, the antibodies and antigen-binding fragments thereof bind to Cynomolgus monkey MASP-2 with a binding affinity similar to that of human MASP-2. For example, binding of the exemplary antibodies 129C10 HaLa, 129C10 HaLb, 129C10 HbLa, 129C10 HbLb, 129C10 HcLa, 129C10 HcLb, 129C10 HdLa, or 129C10 HdLb antibodies to Cynomolgus monkey MASP-2 is at a similar affinity or EC₅₀ value to that of human MASP-2.

In certain embodiments, the antibodies and the antigen-binding fragments thereof provided herein specifically bind to Cynomolgus monkey MASP-2 expressed on a cell at an EC₅₀ of no more than 0.02 μg/mL, no more than 0.015 μg/mL, no more than 0.01 μg/mL, no more than 0.005 μg/mL, no more than 0.001 μg/mL, by ELISA assay.

In certain embodiments, the antibodies and the antigen-binding fragments thereof provided herein specifically bind to Cynomolgus monkey MASP-2 expressed on a cell at an EC₅₀ of no more than 0.02 μg/mL, no more than 0.015 μg/mL, no more than 0.01 μg/mL, no more than 0.005 μg/mL, no more than 0.001 g/mL, by ELISA assay.

In certain embodiments, the antibodies or antigen-binding fragments thereof provided herein have longer (e.g. at least 5%, 10%, 15%, 20% longer) serum half-life in monkey as compared with OMS721.

OMS721 (hIgG4 kappa), also named as OMS721-analog, is a benchmark antibody having the heavy chain and light chain variable regions of OMS721 (Narsoplimab) from U.S. Pat. No. 9,011,860B2, but having the constant region of human IgG4. OMS721 (hIgG4 kappa) comprises a heavy chain of SEQ ID NO: 32, and a light chain of SEQ ID NO: 33.

Heavy chain of OMS721 (hIgG4 kappa) (SEQ ID NO: 32):
QVTLKESGPVLVKPTETLTLTCTVSGFSLSRGKMGVSWIRQPPGKALEWLAHIFSSDE
KSYRTSLKSRLTISKDTSKNQVVLTMTNMDPVDTATYYCARIRRGGIDYWGQGTLV
TVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Light chain of OMS721 (hIgG4 kappa) (SEQ ID NO: 33):
QPVLTQPPSLSVSPGQTASITCSGEKLGDKYAYWYQQKPGQSPVLVMYQDKORPSGI
PERFSGSNSGNTATLTISGTQAMDEADYYCQAWDSSTAVFGGGTKLTVLRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC

In certain embodiments, the antibodies or antigen-binding fragments thereof provided herein do not cross-react with mouse or rat MASP-2.

In certain embodiments, the antibodies or antigen-binding fragments thereof provided herein do not cross-react with C1s, C1r, MASP1 or MASP3.

In certain embodiments, the antibodies or antigen-binding fragments thereof provided herein is capable of selectively blocking the MBL pathway complement activation and is not involved in classical pathway or alternative pathway.

The antibodies or antigen-binding fragments thereof provided herein can be a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g. glycosylated antibody). A recombinant antibody is an antibody prepared in vitro using recombinant methods rather than in animals.

### Antibody Variants

The present disclosure also encompass various types of variants of the antibodies and antigen-binding fragments thereof provided herein. In certain embodiments, the present disclosure encompasses variants of an exemplary antibody provided herein, i.e., 129C10 mouse/chimeric, 160D10 mouse/chimeric, 125D5 mouse/chimeric, 129C10 HaLa, 129C10 HaLb, 129C10 HbLa, 129C10 HbLb, 129C10 HcLa, 129C10 HcLb, 129C10 HdLa, or 129C10 HdLb antibodies.

In certain embodiments, the antibody variants comprise one or more modifications or substitutions in 1, 2, or 3 CDR sequences as provided in Table 1, the heavy or light chain variable region sequences provided in Table 2, and/or the constant region (e.g., Fc region). Such antibody variants retain specific binding affinity to MASP-2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g., one or more introduced cysteine residues).

A parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variant

An affinity variant may contain modifications or substitutions in one or more CDR sequences as provided in Table 1, or the heavy or light chain variable region sequences provided in Table 2. The affinity variants retain specific binding affinity to MASP-2 of the parent antibody, or even have improved MASP-2 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences in Table 1 and FR sequences provided herein, one or more amino acid residues may be substituted yet the resulting antibody or antigen-binding fragment still retain the binding affinity to MASP-2, or even have an improved binding affinity. Various methods known in the art can be used to achieve this purpose. For example, a library of antibody variants (such as Fab or scFv variants) can be generated and expressed with phage display technology, and then screened for the binding affinity to human MASP-2. For another example, computer software can be used to virtually simulate the binding of the antibodies to human MASP-2, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-MASP-2 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to MASP-2 at a level similar to or even higher than its parental antibody.

In certain embodiments, the anti-MASP-2 antibodies and antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) listed in SEQ ID NO: 15, SEQ ID NO: 17, SEQ ID NO: 18, SEQ ID NO: 20, SEQ ID NO: 22, SEQ ID NO: 24, SEQ ID NO: 26, SEQ ID NO: 16, SEQ ID NO: 19, SEQ ID NO: 28, SEQ ID NO: 30 and in the meantime retain the binding affinity to MASP-2 at a level similar to or even higher than its parent antibody. In some embodiments, a total of 1 to 10 amino acids have been substituted, inserted, or deleted in a sequence selected from the group consisting of SEQ ID NO: 15, SEQ ID NO: 17, SEQ ID NO: 18, SEQ ID NO: 20, SEQ ID NO: 22, SEQ ID NO: 24, SEQ ID NO: 26, SEQ ID NO: 16, SEQ ID NO: 19, SEQ ID NO: 28, SEQ ID NO: 30. In some embodiments, the substitutions, insertions, or deletions occur in regions outside the CDRs (i.e., in the FRs).

### Glycosylation Variant

The anti-MASP-2 antibodies and antigen-binding fragments provided herein also encompass a glycosylation variant, which can be obtained to either increase or decrease the extent of glycosylation of the antibody or antigen binding fragment.

The anti-MASP-2 antibody or antigen binding fragment thereof may comprise one or more amino acid residues with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Fc Variant

The anti-MASP-2 antibodies and antigen-binding fragments provided herein also encompass an Fe variant, which comprises one or more amino acid residue modifications or substitutions at its Fc region and/or hinge region.

In certain embodiments, the anti-MASP-2 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the anti-MASP-2 antibodies or antigen-binding fragments thereof comprise one or more amino acid substitution(s) that increases the serum half-life of antibodies by enhancing their binding affinity to FcRn. For example, the heavy chain constant region comprises one or more amino acid substitutions relative to a wild-type human IgG constant region at amino acid residue 252, 254 or 256 (see U.S. Pat. No. 7,083,784). In certain embodiments, the amino acid substitution at amino acid residue 252 is a substitution with tyrosine, the amino acid substitution at amino acid residue 254 is a substitution with threonine, and an amino acid substitution at amino acid residue 256 is a substitution with glutamic acid (M252Y/S254T/T256E, or YTE). In certain embodiments, the anti-MASP-2 antibodies or antigen-binding fragments thereof comprises a heavy chain constant region having at least 80% (or e.g., at least 85%, 88%, 90%, 91%, 92%, 93%, 94%, 95%, 96%, 97%, 98% or 99%) sequence identity of the wild-type heavy chain constant region, provided that the M252Y/S254T/T256E substitutions are maintained.

In certain embodiments, the one or more amino acid substitution(s) are M252Y/S254T/T256E in the heavy chain of human IgG4 Fc region. In certain embodiments, the heavy chain constant region of human IgG4 with YTE modification has an amino acid sequence of SEQ ID NO: 35 (see Table 2-3).

In certain embodiments, the anti-MASP-2 antibodies and antigen-binding fragments thereof provided herein comprises a heavy chain constant region of human IgG4 and a light chain constant region of human IgG. The heavy chain constant region of human IgG4 has an amino acid sequence of SEQ ID NO: 35, and the light chain constant region has an amino acid sequence of SEQ ID NO: 34.

In certain embodiments, the anti-MASP-2 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Provided herein are also anti-MASP-2 antigen-binding fragments. Various types of antigen-binding fragments are known in the art and can be developed based on the anti-MASP-2 antibodies provided herein, including for example, the exemplary antibodies whose CDR are shown in Table 1, and their different variants (such as affinity variants, glycosylation variants, Fc variants and so on).

In certain embodiments, an anti-MASP-2 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxy terminus to provide for a fusion protein (see, for example, Antibody Engineering, ed. Borrebaeck).

In certain embodiments, the antibodies and antigen-binding fragments thereof can be used as the basis of bispecific or multivalent antibodies.

### Bispecific Antibodies, Multivalent Antibodies

In certain embodiments, the antibodies and antigen-binding fragments thereof provided herein are bivalent, tetravalent, hexavalent, or multivalent. In certain embodiments, the antibodies and antigen-binding fragments thereof provided herein are monospecific, or bispecific.

The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. A bivalent molecule can be monospecific if the two binding sites are both for specific binding of the same antigen or the same epitope. Similarly, a trivalent molecule can be bispecific, for example, when two binding sites are monospecific for a first antigen (or epitope) and the third binding site is specific for a second antigen (or epitope).

In certain embodiments, the antibodies and antigen-binding fragments thereof provided herein can be monospecific but bivalent, trivalent, or tetravalent, with at least two binding sites specific for the same antigen or epitope. This, in certain embodiments, provides for stronger binding to the antigen or the epitope than a monovalent counterpart. In certain embodiments, in a bivalent antigen-binding moiety, the first valent of binding site and the second valent of binding site are structurally identical (i.e. having the same sequences), or structurally different (i.e. having different sequences albeit with the same specificity).

In certain embodiments, the antibodies and antigen-binding fragments thereof provided herein are bispecific. In some embodiments, the bispecific antibodies and antigen-binding fragments thereof provided herein has a first specificity for MASP-2, and a second specificity. In some embodiments, the second specificity is for MASP-2 but to different epitopes. In some embodiments, the second specificity is for a second antigen different from MASP-2.

The bispecific antibodies and antigen-binding fragments provided herein can be made with any suitable methods known in the art. In a conventional approach, two immunoglobulin heavy chain-light chain pairs having different antigenic specificities can be co-expressed in a host cell to produce bispecific antibodies in a recombinant way (see, for example, Milstein and Cuello, Nature, 305: 537 (1983)), followed by purification by affinity chromatography.

Recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g., Gruber et al., J. Immunol., 152:5368 (1994).)

In another method, leucine zipper peptides from the Fos and Jun proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980; WO 91/00360, WO 92/00373, and EP 03089). For another example, the two antibodies or antigen-binding fragments can be cross-linked by conventional methods known in the art, for example, as disclosed in U.S. Pat. No. 4,676,980.

Bispecific antigen-binding fragments may be generated from a bispecific antibody, for example, by proteolytic cleavage, or by chemical linking. For example, an antigen-binding fragment (e.g., Fab′) of an antibody may be prepared and converted to Fab′-thiol derivative and then mixed and reacted with another converted Fab′ derivative having a different antigenic specificity to form a bispecific antigen-binding fragment (see, for example, Brennan et al., Science, 229: 81 (1985)).

In certain embodiments, the bispecific antibody or antigen-binding fragments may be engineered at the interface so that a knob-into-hole association can be formed to promote heterodimerization of the two different antigen-binding sites. “Knob-into-hole” as used herein, refers to an interaction between two polypeptides (such as Fc), where one polypeptide has a protuberance (i.e. “knob”) due to presence of an amino acid residue having a bulky side chain (e.g., tyrosine or tryptophan), and the other polypeptide has a cavity (i.e. “hole”) where a small side chain amino acid residue resides (e.g., alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

### Conjugates

In some embodiments, the anti-MASP-2 antibodies and antigen-binding fragments thereof are linked to one or more conjugate moieties. A conjugate is a moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies or antigen binding fragments thereof are linked to one or more conjugates via a linker. In certain embodiments, the linker is a hydrazone linker, a disulfide linker, a bifunctional linker, dipeptide linker, glucuronide linker, a thioether linker.

In certain embodiments, the anti-MASP-2 antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

The conjugate can be a clearance-modifying agent, therapeutic agent (e.g., a chemotherapeutic agent), a toxin, a radioactive isotope, a detectable label (e.g., a lanthanide, a luminescent label, a fluorescent label, or an enzyme-substrate label), a pharmacokinetic modifying moiety, a DNA-alkylator, a topoisomerase inhibitor, a tubulin-binders, other anticancer drugs called such as androgen receptor inhibitor.

Examples of detectable label may include a fluorescent labels (e.g., fluorescein, rhodamine, dansyl, phycoerythrin, or Texas Red), enzyme-substrate labels (e.g., horseradish peroxidase, alkaline phosphatase, luceriferases, glucoamylase, lysozyme, saccharide oxidases or β-D-galactosidase), radioisotuopes, other lanthanides, luminescent labels, chromophoric moiety, digoxigenin, biotin/avidin, a DNA molecule or gold for detection.

Examples of radioisotopes may include ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸y, ⁹⁰Y, ¹⁷⁷Lu, ²¹¹At, ¹⁸⁶Re, ¹⁸⁸Re, ¹⁵³Sm, ²¹²Bi, and ³²P. Radioisotope labelled antibodies are useful in receptor targeted imaging experiments.

In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like.

In certain embodiments, the conjugate can be a purification moiety such as a magnetic bead or a nanoparticle.

### Polynucleotides and Recombinant Methods

The present disclosure provides isolated polynucleotides that encode the anti-MASP-2 antibodies and antigen-binding fragments thereof. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-MASP-2 antibodies and antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

In some embodiments, the vector system includes mammalian, bacterial, yeast systems, etc, and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMD18-T, pMONO, pSELECT, pUNO, pDUO, Psg5L, pBABE, pWPXL, pBI, p15TV-L, pProl8, pTD, pRS420, pLexA, pACT2.2 etc, and other laboratorial and commercially available vectors. Suitable vectors may include, plasmid, or viral vectors (e.g., replication defective retroviruses, adenoviruses and adeno-associated viruses).

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-MASP-2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is CHO cell.

Host cells are transformed with the above-described expression or cloning vectors for anti-MASP-2 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The anti-MASP-2 antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the anti-MASP-2 antibodies or antigen-binding fragments thereof or the antibody-drug conjugate provided herein, and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcellulose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrrolidone, sodium saccharine, cellulose, magnesium carbonate, etc.

In certain embodiments, the pharmaceutical compositions are formulated into an injectable composition. The injectable pharmaceutical compositions may be prepared in any conventional form, such as liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbitol, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-MASP-2 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Methods of Use

The present disclosure also provides methods of treatment comprising: administering a therapeutically effective amount of the antibody or antigen-binding fragment as provided herein to a subject in need thereof, thereby treating or preventing a MASP-2 dependent complement activation related disease or condition.

MASP-2 (MBL-associated serine protease 2) is involved in the complement system and activates complement system. When MBL binds to a pathogen, MASP-2 is activated to cleave complement components C4 and C2 into C4a, C4b, C2a, and C2b, generating C3 convertase C4bC2b, subsequently C3 being converted to C3b by C4bC2b and finally form membrane attack complex (MAC) after C5 being converted to C5b by C3b. Activation of C3 finally leads to the formation of MAC, and then initiates a series of cascade activation processes of downstream complement system to stimulate innate immune response.

Accordingly, inhibition of MASP-2 could be useful in inhibiting MASP-2 dependent complement activation.

The present disclosure provides methods of inhibiting MASP-2 dependent complement activation in a subject in need thereof, or methods of treating or preventing a condition or a disease associated with MASP-2 dependent complement activation, the methods comprising administering a therapeutically effective amount of the antibody or antigen-binding fragment as provided herein to the subject.

In another aspect, methods are provided to treat a condition in a subject that would benefit from inhibition of MASP-2 dependent complement activation, comprising administering a therapeutically effective amount of the antibody or antigen-binding fragment as provided herein to a subject in need thereof.

In another aspect, the present disclosure further provides a method of reducing level of serum C4 level in a subject, comprising administering to the subject a therapeutically effective amount of the antibody or antigen binding fragment thereof provided herein, thereby reducing the level of serum C4 in the subject.

In another aspect, the present disclosure further provides a method of treating a condition in a subject that would benefit from reduction of serum C4 level in a subject, or treating or preventing a condition or a disease associated with abnormal (e.g. elevated) serum C4 level, comprising administering to the subject a therapeutically effective amount of the antibody or antigen binding fragment thereof provided herein, thereby treating the condition.

In some embodiments, the condition or disease associated with MASP-2 dependent complement activation is endothelial injury related disease.

In some embodiments, the condition or disease associated with MASP-2 dependent complement activation is autoantibody immune complex induced disease. Examples of autoantibody immune complex induced disease include, such as, IgA nephropathy, systemic lupus erythematosus (SLE), Lupus nephritis, thrombotic microangiopathies (TMAs), or hematopoietic transplant-associated thrombotic microangiopathy (TA-TMA).

In some embodiments, the MASP-2 dependent complement activation related disease or condition includes IgA nephropathy, (see for details, e.g., Drachenberg C. B., et. al., Kidney International Reports, 2019, 4(11); Espinosa M, et. al., Clin J Am Soc Nephrol, 2014, 9; Yeo S. C. et. al., Pediatr Nephrol, 2018, 33), lupus nephritis (see, e.g., Gatenby, P. A. Autoimmunity, 1991, 11), thrombotic microangiopathies (TMAs) (see, e.g., Elhadad S. et. al., Clin Exp Immunol. 2021, 203(1)), systemic lupus erythematosus (SLE) (see, e.g., Walport, M. J., Davies, et al., Ann. N.Y Acad Sci. § 75:267-81, 1997), vasculitis (see, e.g., Moake X L., N Engl J Med., 2002, 347), Kawasaki's disease (arteritis) (see, e.g., Nakamura A. et. al., Clin Immunol., 2014, 153(1)), SARS-CoV, MERS-CoV and SARS-CoV-2 (Covid-19) (see, e.g., Gao T. et. al., Highly pathogenic coronavirus N protein aggravates lung injury by MASP-2-mediated complement over-activation. Preprint from medRxiv, 30 Mar. 2020).

In some embodiment, the MASP-2 dependent complement activation related disease or condition is an autoimmune disease, a vascular condition, an ischemia-reperfusion injury, atherosclerosis, an inflammation, a pulmonary condition, extracorporeal reperfusion procedure, a musculoskeletal condition, a renal condition, a skin condition, an organ or tissue transplant procedure, a nervous system disorder or injury, a blood disorder, urogenital condition, a nonobese diabetes or a complication associated with Type 1 or Type 2 diabetes, cancer, endocrine disorder, or an ophthalmologic condition.

In certain embodiments, the condition or disease associated MASP-2 dependent complement activation is an autoimmune disease.

In some embodiments, the autoimmune disease comprises thrombotic microangiopathies (TMAs), atypical hemolytic uremic syndrome (aHUS), hematopoietic transplant-associated thrombotic microangiopathy (TA-TMA), lupus nephritis, systemic lupus erythematosus (SLE) and IgA nephropathy.

In some embodiments, the vascular condition comprises a cardiovascular condition, a cerebrovascular condition, a peripheral (e.g., musculoskeletal) vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy and percutaneous transluminal coronary angioplasty (PTCA).

In some embodiments, the ischemia-reperfusion injury comprises an ischemia-reperfusion injury associated with aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures.

In some embodiments, the inflammation comprises inflammatory gastrointestinal disorder comprising pancreatitis, Crohn's disease, ulcerative colitis, irritable bowel syndrome and diverticulitis.

In some embodiments, the pulmonary condition comprises acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, emphysema, cystic fibrosis, SARS-CoV, MERS-CoV and SARS-CoV-2(Covid-19) related condition.

In some embodiments, the extracorporeal reperfusion procedure comprises hemodialysis, plasmapheresis, leukapheresis, extracorporeal membrane oxygenator (ECMO), heparin-induced extracorporeal membrane oxygenation LDL precipitation (HELP) and cardiopulmonary bypass (CPB).

In some embodiments, the musculoskeletal condition comprises osteoarthritis, rheumatoid arthritis, juvenile rheumatoid arthritis, gout, neuropathic arthropathy, psoriatic arthritis, spondyloarthropathy, crystalline arthropathy and systemic lupus erythematosus (SLE).

In some embodiments, the renal condition comprises mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis and IgA nephropathy.

In some embodiments, the skin condition comprises psoriasis, autoimmune bullous dermatoses, eosinophilic spongiosis, bullous pemphigoid, Epidermolysis bullosa acquisita (EBA), herpes gestationis, thermal burn injury and chemical burn injury.

In some embodiments, the organ or tissue transplant procedure comprises organ allotransplantation, organ xenotransplantation organ and tissue graft.

In some embodiments, the nervous system disorder or injury comprises multiple sclerosis, myasthenia gravis, Huntington's disease, amyotrophic lateral sclerosis, Guillain Barre syndrome, reperfusion following stroke, degenerative discs, cerebral trauma, Parkinson's disease, Alzheimer's disease, Miller-Fisher syndrome, cerebral trauma and/or hemorrhage, demyelination and meningitis.

In some embodiments, the blood disorder comprises sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome.

In some embodiments, the urogenital condition comprises painful bladder disease, sensory bladder disease, chronic abacterial cystitis, interstitial cystitis, infertility, placental dysfunction and miscarriage and pre-eclampsia.

In some embodiments, the endocrine disorder comprises Hashimoto's thyroiditis, stress, anxiety and hormonal disorders involving regulated release of prolactin, growth or other insulin-like growth factor and adrenocorticotropin from the pituitary.

In some embodiments, the ophthalmologic condition comprises age-related macular degeneration.

In some embodiments of the methods provided herein, the subject is determined to have an elevated C4 serum level, or have C4d deposition or C4d positive staining in a sample of interest. In some embodiments, the condition or disease is IgA nephropathy. C4d-positive staining has been reported as an independent risk factor for the development of ESRD in IgAN. C4d can be detected using any suitable methods known in the art, for example, by using ELISA, or immunofluorescence microscopy.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, an anti-MASP-2 antibody or antigen-binding fragment as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The anti-MASP-2 antibodies and antigen-binding fragments disclosed herein may be administered by any route known in the art, such as parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the anti-MASP-2 antibodies or antigen-binding fragments disclosed herein may be administered alone or in combination with one or more additional therapeutic means or agents. For example, the antibodies or antigen-binding fragments disclosed herein may be administered in combination with another therapeutic agent, for example, an anti-autoimmune drug.

In certain of these embodiments, an anti-MASP-2 antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an anti-MASP-2 antibody or antigen-binding fragment administered “in combination” with another therapeutic agent does not have to be administered simultaneously with or in the same composition as the agent. An anti-MASP-2 antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In some embodiments, the present disclosure also provides use of the antibody or antigen-binding fragment thereof provided herein in the manufacture of a medicament for treating a MASP-2 dependent complement activation related disease or condition in a subject.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1: Generation of Human, Mouse, Cynomolgus MASP-2 Antigens

### 1. Construction of Antigen for Expression

To limit antibodies' epitope to the complement binding and activation domains of human MASP-2, a chimeric antigen for animal immunization was designed. The coding sequences expressing mouse MASP-2 CUB1-EGF-CUB2 domain (residues 20-297 of SEQ ID NO: 40) and human MASP-2 CCP1-CCP2-SP domain (residues 298-686 of SEQ ID NO: 39) were synthesized. In addition, an IL-2 secretion signal peptide sequence (SEQ ID NO: 41) was added at the N terminal and a FLAG-tag sequence was added at the C terminal. The above elements were combined into one open reading frame (ORF) to make a chimeric antigen expression construct (SEQ ID NO: 42). Other constructs expressing the full-length human MASP-2 (SEQ ID NO: 39), mouse MASP-2 (SEQ ID NO: 40) and cynomolgus MASP-2 (SEQ ID NO: 43), with a HIS-tag at the C terminal, were made by synthesis. Also, another construct expressing the full length human MASP-2 (SEQ ID NO: 39) with a FLAG-tag at the C terminal was made by PCR.

Human MASP-2 protein
(SEQ ID NO: 39)
MRLLTLLGLLCGSVATPLGPKWPEPVFGRLASPGFPGEYANDQERRWTLTAPPGYRL
RLYFTHFDLELSHLCEYDFVKLSSGAKVLATLCGQESTDTERAPGKDTFYSLGSSLDI
TFRSDYSNEKPFTGFEAFYAAEDIDECQVAPGEAPTCDHHCHNHLGGFYCSCRAGYV
LHRNKRTCSALCSGQVFTQRSGELSSPEYPRPYPKLSSCTYSISLEEGFSVILDFVESFD
VETHPETLCPYDFLKIQTDREEHGPFCGKTLPHRIETKSNTVTITFVTDESGDHTGWKI
HYTSTAQPCPYPMAPPNGHVSPVQAKYILKDSFSIFCETGYELLQGHLPLKSFTAVCQ
KDGSWDRPMPACSIVDCGPPDDLPSGRVEYITGPGVTTYKAVIQYSCEETFYTMKVN
DGKYVCEADGFWTSSKGEKSLPVCEPVCGLSARTTGGRIYGGQKAKPGDFPWQVLI
LGGTTAAGALLYDNWVLTAAHAVYEQKHDASALDIRMGTLKRLSPHYTQAWSEAV
FIHEGYTHDAGFDNDIALIKLNNKVVINSNITPICLPRKEAESFMRTDDIGTASGWGLT
QRGFLARNLMYVDIPIVDHQKCTAAYEKPPYPRGSVTANMLCAGLESGGKDSCRGD
SGGALVFLDSETERWFVGGIVSWGSMNCGEAGQYGVYTKVINYIPWIENIISDF
Mouse MASP-2 protein
(SEQ ID NO: 40)
MRLLIFLGLLWSLVATLLGSKWPEPVFGRLVSPGFPEKYADHQDRSWTLTAPPGYRL
RLYFTHFDLELSYRCEYDFVKLSSGTKVLATLCGQESTDTEQAPGNDTFYSLGPSLK
VTFHSDYSNEKPFTGFEAFYAAEDVDECRVSLGDSVPCDHYCHNYLGGYYCSCRAG
YVLHQNKHTCSALCSGQVFTGRSGYLSSPEYPQPYPKLSSCTYSIRLEDGFSVILDFVE
SFDVETHPEAQCPYDSLKIQTDKGEHGPFCGKTLPPRIETDSHKVTITFATDESGNHTG
WKIHYTSTARPCPDPTAPPNGSISPVQAIYVLKDRFSVFCKTGFELLQGSVPLKSFTAV
CQKDGSWDRPMPECSIIDCGPPDDLPNGHVDYITGPEVTTYKAVIQYSCEETFYTMSS
NGKYVCEADGFWTSSKGEKLPPVCEPVCGLSTHTIGGRIVGGQPAKPGDFPWQVLLL
GQTTAAAGALIHDNWVLTAAHAVYEKRMAASSLNIRMGILKRLSPHYTQAWPEEIFI
HEGYTHGAGFDNDIALIKLKNKVTINGSIMPVCLPRKEAASLMRTDFTGTVAGWGLT
QKGLLARNLMFVDIPIADHQKCTAVYEKLYPGVRVSANMLCAGLETGGKDSCRGDS
GGALVFLDNETQRWFVGGIVSWGSINCGAADQYGVYTKVINYIPWIENIISNF
IL-2 secretion signal peptide sequence
(SEQ ID NO: 41)
MYRMQLLSCIALSLALVINS
Chimeric MASP-2 protein
(SEQ ID NO: 42)
MYRMQLLSCIALSLALVINSSKWPEPVFGRLVSPGFPEKYADHQDRSWTLTAPPGYR
LRLYFTHFDLELSYRCEYDFVKLSSGTKVLATLCGQESTDTEQAPGNDTFYSLGPSLK
VTFHSDYSNEKPFTGFEAFYAAEDVDECRVSLGDSVPCDHYCHNYLGGYYCSCRAG
YVLHQNKHTCSALCSGQVFTGRSGYLSSPEYPQPYPKLSSCTYSIRLEDGFSVILDFVE
SFDVETHPEAQCPYDSLKIQTDKGEHGPFCGKTLPPRIETDSHKVTITFATDESGNHTG
WKIHYTSTAQPCPYPMAPPNGHVSPVQAKYILKDSFSIFCETGYELLQGHLPLKSFTA
VCQKDGSWDRPMPACSIVDCGPPDDLPSGRVEYITGPGVTTYKAVIQYSCEETFYTM
KVNDGKYVCEADGFWTSSKGEKSLPVCEPVCGLSARTTGGRIYGGQKAKPGDFPW
QVLILGGTTAAGALLYDNWVLTAAHAVYEQKHDASALDIRMGTLKRLSPHYTQAW
SEAVFIHEGYTHDAGFDNDIALIKLNNKVVINSNITPICLPRKEAESFMRTDDIGTASG
WGLTQRGFLARNLMYVDIPIVDHQKCTAAYEKPPYPRGSVTANMLCAGLESGGKDS
CRGDSGGALVFLDSETERWFVGGIVSWGSMNCGEAGQYGVYTKVINYIPWIENIISD
FDYKDDDDK
Cynomolgus MASP-2 protein
(SEQ ID NO: 43)
MRLLTLLGLLCGSVATPLGPKWPEPVFGRLASPGFPGEYANDQERRWTLTAPPGYRL
RLYFTHFDLELSHLCEYDFVKLSSGAKVLATLCGHESTDTERAPGNDTFYSLGSSLDI
TFRSDYSNEKPFTGFEAFYAAEDIDECQVAPGEAPACDHHCHNHLGGFYCSCRVGYI
LHRNKRTCSALCSGQVFTQRSGELSSPEYPQPYPKLSSCTYSIRLEEGFSVILDFVESFD
VETHPETLCPYDFLKIQIDSEEHGPFCGKTLPRRIETKSNTVTITFVTDESGDHTGWKI
HYTSTAQPCPYPMAPPNGHLSPVQAKYILKDSFSIFCEPGYELLQGHLPLKSFAAVCQ
KDGSWDQPMPSCSIVDCGPPDDLPSGRVEYITGPEVTTYKAVIQYSCEETFYTMKVN
DGKYVCEADGFWTSSKGERSPPVCEPVCGLSARTTGGRIYGGQKAKPGDFPWQVLI
LGGSTAAGALLYDNWVLTAAHAIYEQKHDASSLDIRLGALKRLSPHYTQAWAEAVF
IHEGYTHDAGFDNDIALIKLNNKVVINSNITPICLPRKEAESFMRTDDIGTASGWGLTQ
RGLLARNLMYVDIPIVDHQKCTAAYEKPPYSGGSVTANMLCAGLESGGKDSCRGDS
GGALVFLDNETQRWFVGGIVSWGSMNCGEAGQYGVYTKVINYIPWIKNIISNF

### 2. Expression and Purification of MASP-2 Antigens

The MASP-2 expression constructs as described above were separately transfected into ExpiCHO-s cells with an ExpiFectamine CHO transfection kit. ExpiCHO-s cells were cultured in the ExpiCHO Expression Medium without serum. 14 days post transfection, the supernatants were collected. After centrifuge and filtration, supernatants were loaded onto anti-FLAG or anti-HIS column and then purified with a GE AKTA purification system. After washing, MASP-2 proteins were eluted with citric acid (pH3.5) for animal immunization.

### Example 2: MASP-2 Antibody Generation

### 1. Immunization and Hybridoma Fusion

Mice or rats of different lineages were immunized with DNA that expressing the chimeric MASP-2 antigen described above via a Helios gene gun system (Bio-Rad). The immunized animals were boosted with the recombinant MASP-2 protein every two weeks. The animals were sacrificed for hybridoma fusion 4 days after the final boost. Spleen cells were isolated and fused with SP2-0 cells via an electro-fusion method. The resulting hybridoma cells were cultured in DMEM medium containing hypoxanthine-aminopterin-thymidine.

### 2. Hybridoma Screening

After 10-days culture, hybridoma supernatants were collected for antigen binding screening. Full-length human MASP-2 protein was coated at a concentration of 0.5 μg/ml 100 μl per well in ELISA plate. After blocking with PBS containing 1% BSA+1% normal goat serum+0.05% Tween20, the hybridoma supernatants were added to the plate for 1 hr. The specific binding of the hybridoma antibodies to human MASP-2 was detected with a horseradish peroxidase (HRP)-linked anti-mouse antibody. The ELISA binding positive clones were selected and proceeded to further activity screening.

### 3. Activity Screening

ELISA plates were coated with 10 μg/ml mannan 100 μl per well at 4° C. for overnight. After washing 3 times with PBS+0.1% Tween20, the plates were blocked for 1 hr with the blocking buffer (10 mM Tris-HCl+0.1% human serum albumin+140 mM NaCl). 50 μl of the hybridoma supernatants were mixed with 50 μl of 1% human serum (Quidel, A113) diluted with the assay buffer (0.1% human serum albumin+20 mM Tris-HCl+2 mM CaCl₂+140 mM NaCl+1 mM MgCl₂+0.05% Tween20) and then incubated on ice for 45 min. Blocking buffer was removed from the mannan coated plates, and the supernatant-serum mixture were added. The plates were incubated at 37° C. for 1.5 hr. After washing 3 times with the washing buffer, activation of C4 was monitored by measuring C4b deposition. The deposited C4b was detected by an HRP-linked anti-C4c antibody (Quidel-A211). If the activity of MASP-2 is inhibited by antibody, there is less C4b deposited on the bottom of the plate. By using this method, the hybridoma antibodies with MASP-2 inhibition activity were selected.

The positive antibodies were then subjected to subclone and re-screened with both ELISA binding and C4 activation assays.

### Example 3: Characterization of Inhibition Activity of Anti-Human MASP-2 Antibodies

### 1. Hybridoma Antibodies Purification

After subcloning, the positive hybridoma clones with neutralization activity were expanded in 10 cm dish. After centrifuge and filtration, the antibody-containing supernatant was loaded onto protein A column and purified with the GE AKTA purification system. After washing, antibody was eluted with citric acid (pH3.5).

### 2. Activity Evaluation of Purified Antibodies

MASP-2 is a key component of lectin pathway, and it cleaves complement factors C4 and C2, generating C3 convertase C4bC2a. Activation of C3 finally leads to the formation of membrane attack complex (MAC). To test whether antibodies that inhibit MASP-2 could reduce activation of the lectin pathway, activation of C4, C3 and MAC was evaluated in the presence of the purified MASP-2 antibodies.

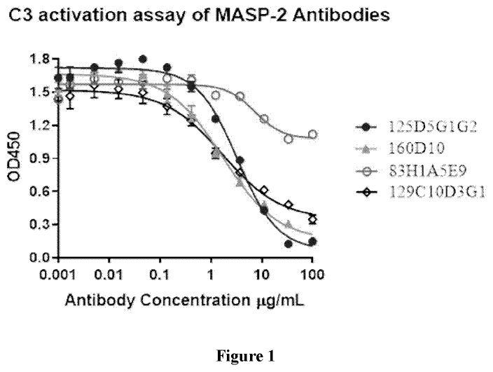

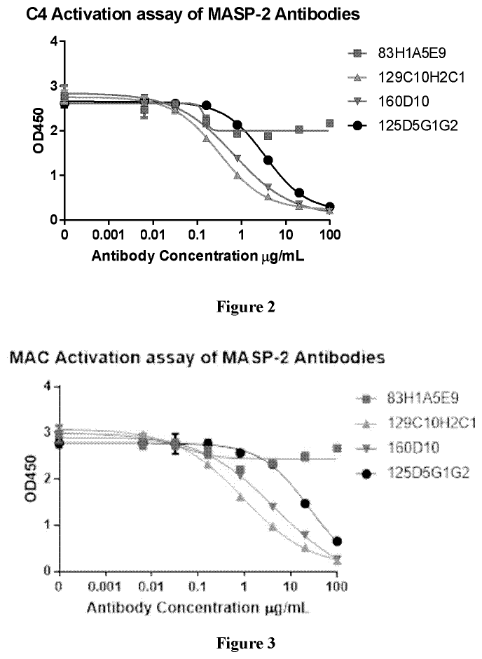

ELISA plates were coated with 10 μg/ml mannan 100 μl per well at 4C for overnight. After washing 3 times with PBS+0.1% Tween20, the plates were blocked for 1 hr with the blocking buffer (10 mM Tris-HCl+0.1% human serum albumin+140 mM NaCl). Antibodies were serially diluted with the assay buffer (0.1% human serum albumin+20 mM Tris-HCl+2 mM CaCl₂+140 mM NaCl+1 mM MgCl₂+0.05% Tween20) containing 1% human serum (Quidel, A113) and incubated on ice for 45 min. The blocking buffer was removed from the mannan coated plates and the antibody-serum mixture was added. The plates were incubated at 37° C. for 1.5 hr. The activated complement components should be deposited on the bottom surface of the plate, while the inactivated components remain soluble in the buffer. After washing for 3 times with the washing buffer, activation of the complement components was monitored by HRP-linked complement antibodies including anti-C3c antibody (Quidel-A205), anti-C4c antibody (Quidel-A211) and anti-MAC (SC5b-9) antibody (Quidel-A239). The detection antibodies were linked with RP in house. The MASP-2 antibodies purified from hybridoma cells blocked the activation of complement C3 (see FIG. 1), complement C4 (see FIG. 2) and MAC (see FIG. 3) in a dose-dependent manner.

### Example 4: V-Gene Cloning and Generation of Chimeric Antibodies

### 1. V-Gene Cloning and Sequencing of Hybridoma Antibodies

The lead antibodies with desired profile were selected for V-gene cloning. The sequences of the murine anti-human MASP-2 light chain and heavy chain variable regions were obtained by the polymerase chain reaction (PCR) amplification technique. Total RNA from the positive hybridoma cell was isolated by using the MiniBest Universal RNA Extraction Kit (TaKaRa), and the cDNA was synthesized by using 1st Strand cDNA Synthesis Kit (TaKaRa) with Oligo (dT) primer. The variable regions of mouse IgG gene were amplified by PCR by using primers of different isotypes for heavy chain variable region and Kappa chain primers for light chain variable region. The PCR products were subcloned into a TA cloning vector. For each variable gene construct, more than 10 single colonies were used for DNA sequencing by Synbio Technologies (Suzhou, China). The amino acid sequences of VH and Vκ were derived from the DNA sequencing results.

### 2. Construction of Chimeric Antibodies

Three antibodies with different sequences, including 129C10, 160D10 and 125D5 were selected as lead antibodies to generate chimeric antibodies, and their SEQ were listed in the Tables 1 and 2. After sequencing analysis and confirmation, cDNAs of the variable regions of heavy chain and light chain were synthesized and fused with the sequences of the constant region of human IgG4 and human kappa. To enhance antibody secretion, the signal peptide sequences were added at N-terminal of heavy chain and light chain, respectively. The resulting chimeric antibody genes were cloned into an expression vector. Large-scale DNA was prepared by using Plasmid Maxi-prep System from Qiagen.

### 3. Expression and Purification of Chimeric Antibodies

Co-transfection of heavy chain and light chain was carried out using the ExpiFectamine™ CHO Reagent from Invitrogen according to the manufacturer's protocol. ExpiCHO-S cells in ExpiCHO Expression Medium at 5-6×10⁶ cell/ml were transfected with equal amount of heavy chain vector and light chain vector DNA at a final concentration of 0.8 μg/ml by using ExpiFectamine™ CHO Reagent. The plasmid DNA or ExpiFectamine™ CHO Reagent was diluted with cold OptiPRO™ medium, and then mixed by swirling tube and/or inversion. The ExpiFectamine™ CHO/plasmid DNA mixtures were incubated at room temperature for 1-5 minutes, and then slowly transferred into a shaker flask with cells. The transfecting cells were incubated at 37° C. with a humidified atmosphere of 5% CO₂ on an orbital shaker (125 rpm shaking speed). 18 to 22 hours after transfection, ExpiCHO™ Feed was added, and the conditioned medium was harvested on day 10. The supernatant was centrifuged at 4,000 rpm for 20 minutes and then filtered through 0.22 m filtration capsule to remove cell debris. The filtered supernatant was loaded onto a pre-equilibrated Protein-A affinity column. Protein-A resin was washed with equilibration buffer (PBS), and 25 mM citrate (pH3.5) was then used to elute the antibody. The purified antibody solution was adjusted to pH 6.0-7.0 by using 1M Tris-base (pH 9.0). The endotoxin was controlled below 1 EU/mg. Finally, the purified antibody was characterized by SDS-PAGE.

### Example 5: Generation and Characterization of Humanized Antibodies

### 1. Generation, Expression and Purification of Humanized Antibodies

The sequences of the variable domains of mouse antibody 129C10 were used to identify the germline sequences with the highest homology to their respective murine framework. Computer-modelling was used for designing the humanized variants with CDR grafting and back mutations.

### 129C10

Human germline framework sequences VH/1-2 for heavy chain and VK/2-30 for light chain were used for CDR grafting, respectively.

Heavy chain (HC) variants 1, 2, 3 and 4 were obtained by direct grafting the three CDRs to the germline sequence (SEQ ID NO: 37), and in addition the mutations of R71V, A93T for HC variant 1 (SEQ ID NO: 20), R71V, A93T, V67A, M69L for HC variant 2 (SEQ ID NO: 22), R71V, A93T, A65G, K64Q, E61Q for HC variant 3 (SEQ ID NO: 24) and R71V, A93T, V67A, M69L, A65G, K64Q, E61Q for HC variant 4 (SEQ ID NO: 26), respectively. It should be noted that there are 3 mutations (A65G, K64Q, E61Q) of HC variants 3 and 4 introduced in HC CDR2, in order to further improve the antibody's humanness.

### Germline Sequence for 129C10 HC:

VH/1-2 (129C10-HC germline, SEQ ID NO: 37):
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
VH/1-2 variant 1 (Hu129C10_Ha, SEQ ID NO: 20):
QVQLVQSGAEVKKPGASVKVSCKASGYTFTDYYINWVRQAPGQGLEWMG
WIFPGSESAYHSEKFKARVTMTVDTSISTAYMELSRLRSDDTAVYYCTR
GDRSGPFAYWGQGTLVTVSS
VH/1-2 variant 2 (Hu129C10_Hb, SEQ ID NO: 22):
QVQLVQSGAEVKKPGASVKVSCKASGYTFTDYYINWVRQAPGQGLEWMG
WIFPGSESAYHSEKFKARATLTVDTSISTAYMELSRLRSDDTAVYYCTR
GDRSGPFAYWGQGTLVTVSS
VH/1-2 variant 3 (Hu129C10_Hc, SEQ ID NO: 24):
QVQLVQSGAEVKKPGASVKVSCKASGYTFTDYYINWVRQAPGQGLEWMG
WIFPGSESAYHSQKFQGRVTMTVDTSISTAYMELSRLRSDDTAVYYCTR
GDRSGPFAYWGQGTLVTVSS
VH/1-2 variant 4 (Hu129C10_Hd, SEQ ID NO: 26):
QVQLVQSGAEVKKPGASVKVSCKASGYTFTDYYINWVRQAPGQGLEWMG
WIFPGSESAYHSQKFQGRATLTVDTSISTAYMELSRLRSDDTAVYYCTR
GDRSGPFAYWGQGTLVTVSS

Light chain (LC) variants 1 and 2 were obtained by direct grafting the three CDRs to the germline sequence (SEQ ID NO: 38), and in addition the back mutations of F36L for LC variant 1 (SEQ ID NO: 28) and F36L, T69A for LC variant 2 (SEQ ID NO: 30), respectively.

### Germline Sequences for 129C10 LC

VK/2-30 (129C10-LC-germline, SEQ ID NO: 38)
DVVMTQSPLSLPVTLGQPASISCRSSQSLVYSDGNTYLNWFQQRPGQSP
RRLIYKVSNRDSGVPDRFSGSGSGTDFTLKISRVEAEDVGVYYCMQGTH
WP
VK/2-30 variant 1 (Hu129C10_La, SEQ ID NO: 28)
DVVMTQSPLSLPVTLGQPASISCKSSQSLLYSNGKTYLNWLQQRPGQSP
RRLIYLVSKLDSGVPDRFSGSGSGTDFTLKISRVEAEDVGVYYCVQVTH
FPFTFGQGTKLEIK
VK/2-30 variant 2 (Hu129C10_Lb, SEQ ID NO: 30)
DVVMTQSPLSLPVTLGQPASISCKSSQSLLYSNGKTYLNWLQQRPGQSP
RRLIYLVSKLDSGVPDRFSGSGSGADFTLKISRVEAEDVGVYYCVQVTH
FPFTFGQGTKLEIK

The cDNAs of the variable regions of the above heavy chains and light chains were synthesized and then fused with the sequences of the constant region of human IgG4 and human kappa. The resulting antibody gene sequences were cloned into an expression vector. Large-scale DNA was prepared by using Plasmid Maxiprep System from Qiagen, and cell transfection was carried out using the ExpiFectamine™ CHO Reagent from Invitrogen according to the manufacturer's protocol. Supernatant was harvested when cell viability was more than 60% and filtered through 0.22 m filtration capsule to remove cell debris. The filtered supernatant was subsequently loaded onto a pre-equilibrated Protein-A affinity column. Protein A resin was washed with equilibration buffer (PBS), and 25 mM citrate (pH3.5) was then used to elute antibody. The purified antibody solution was adjusted to pH 6.0-7.0 by using 1M Tris-base (pH 9.0). The endotoxin was controlled below 1 EU/mg. Finally, the purified antibody was characterized by SDS-PAGE.

Benchmark antibody OMS721-analog and 129C10-hu-YTE (Hu129C10_HaLa-hIgG4 with amino acid substitutions M252Y/S254T/T256E [YTE], see Table 2-3) were also constructed, and expression and purification procedures were same as above.

### Example 6: Blocking Activity of the Chimeric and Humanized MASP-2 Antibodies

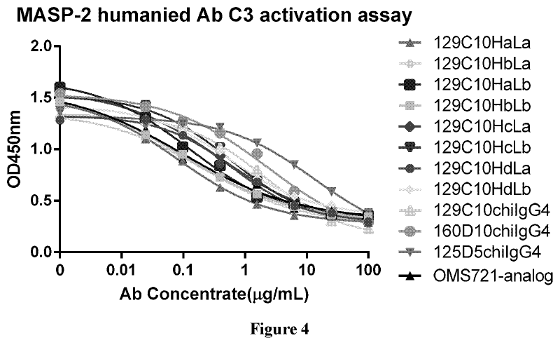

The purified antibodies were serially diluted from 100 μg/ml to get a gradient concentration. The complement C3 activation assay was used to evaluate MASP-2 antibody blocking activity as described in Example 3. 100 μl per well of 10 μg/ml mannan was coated onto ELISA plate at 4° C. for overnight. Antibodies were incubated with 1% human serum (Quidel, A113) for 45 min on ice. After washing and blocking of the plate, the antibody-serum mixture was added and incubated at 37° C. for 90 min. After washing, the deposited activated C3 was detected with an HRP-linked anti-C3c antibody (Quidel-A205). FIG. 4 shows blocking activity of the chimeric and humanized antibodies for activation of the complement C3. As the humanized 129C10 variant, 129C10HaLa, showed best affinity and C3 blocking activity, 129C10HaLa was selected as the lead antibody for further in vitro and in vivo studies and it was named as 129C10-hu.

### Example 7: Activity Comparison of Blocking Complement C4 and MAC Between the Lead Antibody 129C10-Hu and the Benchmark Antibody OMS721-Analog

### 1. Activity Comparison of Blocking Complement C4 and MAC Activation

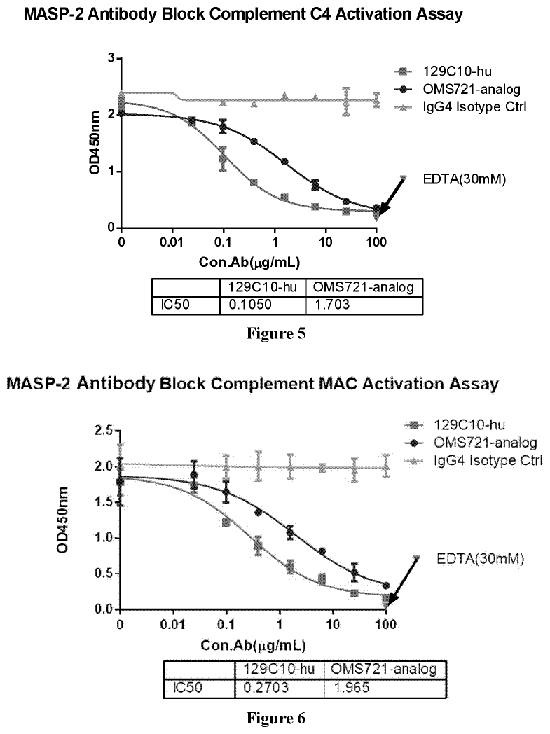

The lead MASP-2 antibody 129C10-hu was compared with OMS721-analog in their blocking activity to complement C4 and MAC activation in 2% human serum using the assay as described in Example 3 with minor changes. 100 μl per well of 10 μg/ml mannan was coated onto ELISA plate at 4° C. for overnight. Antibodies were incubated with 2% human serum (Quidel, A113) for 45 min on ice. After washing and blocking of the plate, the antibody-serum mixture was added and incubated at 37° C. for 90 min. After washing, the deposited activated C4 was detected with an HRP-linked anti-C4c antibody (Quidel-A211). For MAC activation, it was detected with an HRP-linked anti-MAC (SC5b-9) antibody (Quidel-A239). As shown in FIG. 5 and FIG. 6, the results demonstrated that 129C10-hu was around 10-folds more potent than OMS721-analog in blocking activation of complement C4 (IC50: 0.11 μg/mL vs. 1.70 μg/mL) and MAC (IC50: 0.27 μg/mL vs. 1.97 μg/mL).

### 2. Activity Comparison in Different Concentrations of Serum

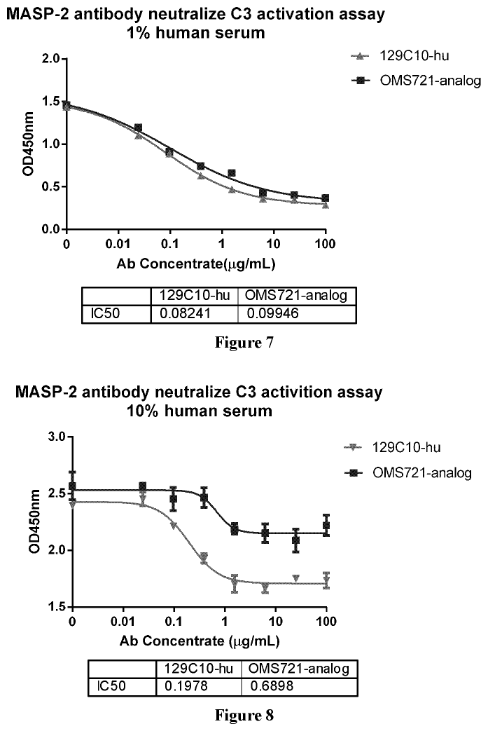

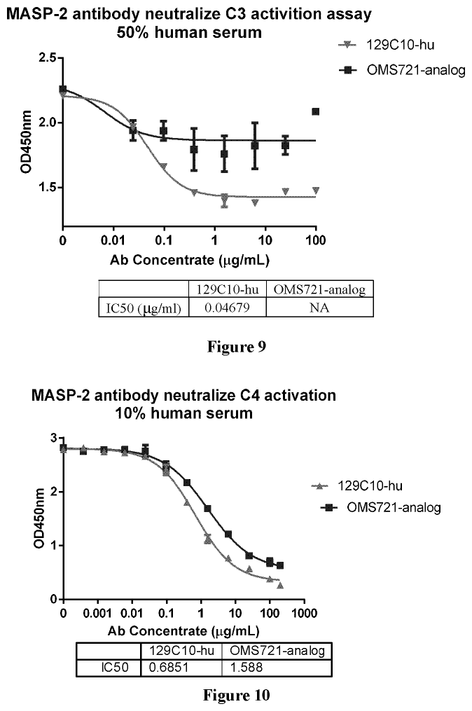

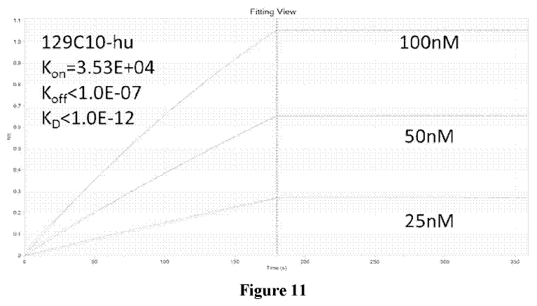

Different concentrations (1%, 10% and 50%) of human serum were used in complement C3 activation assay. For 1% and 10% human serum, the assay method was same as described in Example 3. For 50% human serum, mannan was coated at a concentration of 1 μg/ml, 100 μl per well at 4° C. for overnight. After washing 3 times with PBS+0.1% Tween20, the plates were blocked for 1 hr with blocking buffer (10 mM Tris-HCl+0.1% human serum albumin+140 mM NaCl). Antibodies were serially diluted with assay buffer (0.1% human serum albumin+20 mM Tris-HCl+2 mM CaCl₂+140 mM NaCl+1 mM MgCl₂+0.05% Tween20) containing 50% human serum (Quidel, A113) and then incubated on ice for 45 min. Blocking buffer was removed from mannan coated plates and the antibody-serum mixture were added. The plates were incubated at 37° C. for 30 min. After washing for 3 times, the activated C3 was detected with an RP-linked anti-C3c antibody (Quidel-A205). As shown in FIG. 7, 129C10-hu and OMS721-analog had a similar inhibition efficacy to C3 activation in 1% serum (IC50: 0.08 μg/mL vs. 0.10 μg/mL). Interestingly 129C10-hu demonstrated 3-folds more potent than OMS721-analog at a high concentration of 10% serum (IC50: 0.20 μg/mL vs. 0.69 μg/mL) (see FIG. 8). Even more significantly at a concentration of 50% serum, 129C10-hu remained its blocking activity to C3 activation at an IC50 of 0.05 μg/mL, while OMS721-analog lost its activity (see FIG. 9). For blocking C4 activation, 129C10-hu also showed 2-folds more potent than OMS721-analog in 10% human serum (IC50: 0.69 μg/mL vs. 1.59 μg/mL) (see FIG. 10).

### Example 8: Binding Affinity of MASP-2 Antibodies to Human MASP-2 by Bio-Layer Interferometry (ForteBio)

Antibodies to be tested were diluted with ForteBio kinetics buffer (PBS pH 7.4, 0.1% BSA+0.002% Tween-20) to a concentration of 100 nM. Human MASP-2 protein was diluted with kinetics buffer to get a three-concentration gradient of 100 nM, 50 nM and 25 nM. 0 nM was used as a reference control. Antibodies was immobilized onto Protein A biosensor. The baseline was detected for 60 seconds, and then antibody-MASP-2 association was detected for 180 seconds to get the Kₒₙ factor data. Followed by dissociation in kinetic buffer for 180 seconds to get the K_{off} factor data. The regeneration of biosensors was in a buffer of 10 mM glycine, pH2.0. All the kinetics data were collected at 30° C. Data were acquired by using the ForteBio Octet RED96 and analyzed by using the Octet Data Analysis software. As shown in Table 3, all the tested MASP-2 hybridoma antibodies had a high binding affinity to human MASP-2 with KD values ranging from 10⁻¹⁰ M to 10⁻⁸ M. After humanization, the lead antibody 129C10-hu (129C10-HaLa) kept the highest binding affinity to hMASP-2 at a KD value of <10⁻¹² M (reach the detection limit of ForteBio) (Table 4), with a Kₒₙ value of 3.53E+4 and a K_{off} value of <1.0E-7 (see FIG. 11).

TABLE 3
Binding Affinity of MASP-2 Hybridoma Antibodies

| | Affinity | | |
|---|---|---|---|
| Sample | K_{D} (M) | kₒₙ(1/Ms) | k_{dis}(1/s) |
| 129C10H2C1 | 7.46E−10 | 6.40E+04 | 4.78E−05 |
| 160D10 | 2.78E−08 | 3.61E+04 | 1.00E−03 |
| 77C1H1E1 | 1.18E−09 | 6.96E+04 | 8.20E−05 |
| 128D5G3H2 | 1.05E−09 | 8.39E+04 | 8.81E−05 |
| 125D5G1G2 | 1.64E−09 | 5.94E+04 | 9.73E−05 |
| 160F3E1G5 | 1.41E−09 | 7.09E+04 | 1.00E−04 |
| 80C12G2F10 | 4.49E−09 | 9.25E+04 | 4.15E−04 |
| 115H10B3E4 | 7.20E−10 | 7.29E+04 | 5.24E−05 |

TABLE 4
Binding Affinity of MASP-2 Humanized or Chimeric Antibodies

| Sample | K_{D} (M) | kₒₙ(1/Ms) | k_{dis}(1/s) |
|---|---|---|---|
| 129C10HaLa | <1.0E−12 | 3.53E+04 | <1.0E−07 |
| 129C10HbLa | <1.0E−12 | 3.29E+04 | <1.0E−07 |
| 129C10HbLb | <1.0E−12 | 3.29E+04 | <1.0E−07 |
| 129C10HcLb | 1.36E−09 | 3.37E+04 | 4.56E−05 |
| 129C10HaLb | 1.14E−08 | 2.55E+04 | 2.91E−04 |
| 129C10HdLb | <1.0E−12 | 3.60E+04 | <1.0E−07 |
| 129C10HdLa | 1.71E−08 | 2.49E+04 | 4.25E−04 |
| 129C10HcLa | <1.0E−12 | 3.52E+04 | <1.0E−07 |
| 129C10chiIgG4 | 8.02E−09 | 2.57E+04 | 2.06E−04 |
| 160D10chiIgG4 | 9.84E−10 | 3.40E+04 | 3.34E−05 |
| 125D5chilgG4 | 1.95E−08 | 2.56E+04 | 4.99E−04 |
| OMS721-analog | 7.40E−09 | 4.16E+04 | 3.08E−04 |

### Example 9: Binding Specificity of 129C10-Hu by ELISA

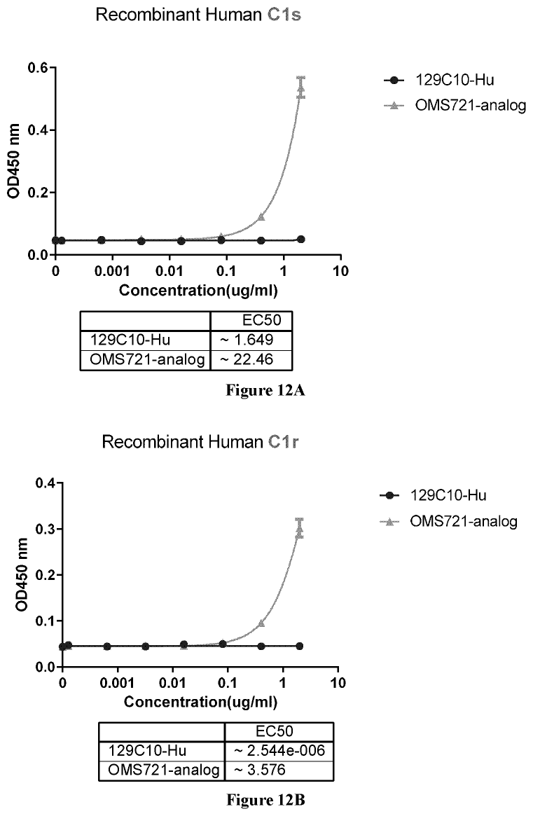

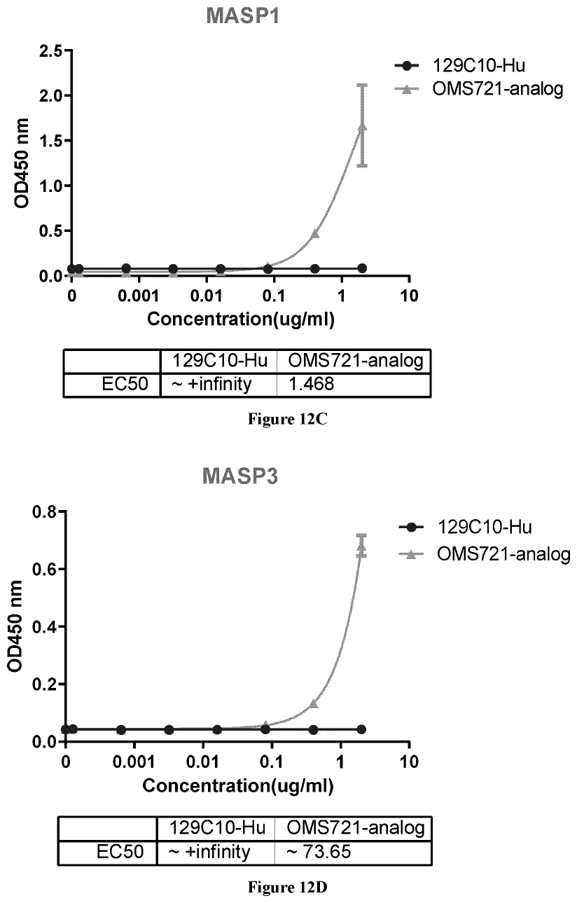

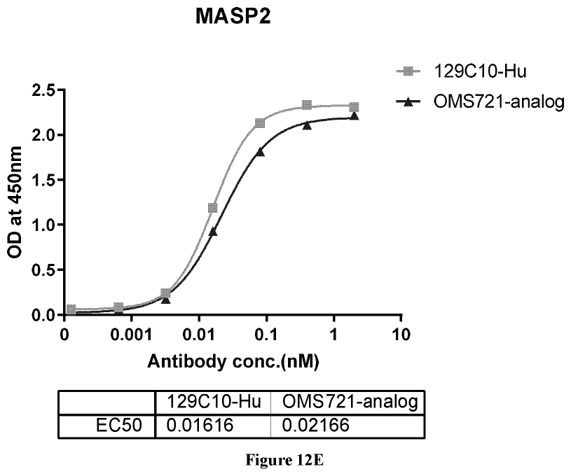

Human C1s/C1r, MASP-1/3 were purchased from R&D, Cusbio etc. Coat Recombinant Human Complement Component C1s/C1r, MASP-1 or MASP-3 (1 mg/ml) at 4° C. for overnight; wash three times with wash buffer; add blocking buffer (200 μL/well) at 4° C. for 1 h; wash three times; add serially diluted Ab at RT for 1 h; wash three times; add mouse anti-human IgG4 HRP (1:20000) at RT for 1 h; wash three times; detected with tetramethylbenzidine (TMB) for 40 min at OD450 nm. The EC50 of binding of 129C10-Hu and OMS721-analog to C1s, C1r, MASP1, MASP2 or MASP3 were shown in FIG. 12A-12E, respectively. The results showed that 129C10-Hu only bound to human MASP2, but not to human C1s, C1r, MASP1 or MASP3.

### Example 10: Cross-Reactivity of 129C10-Hu by ELISA

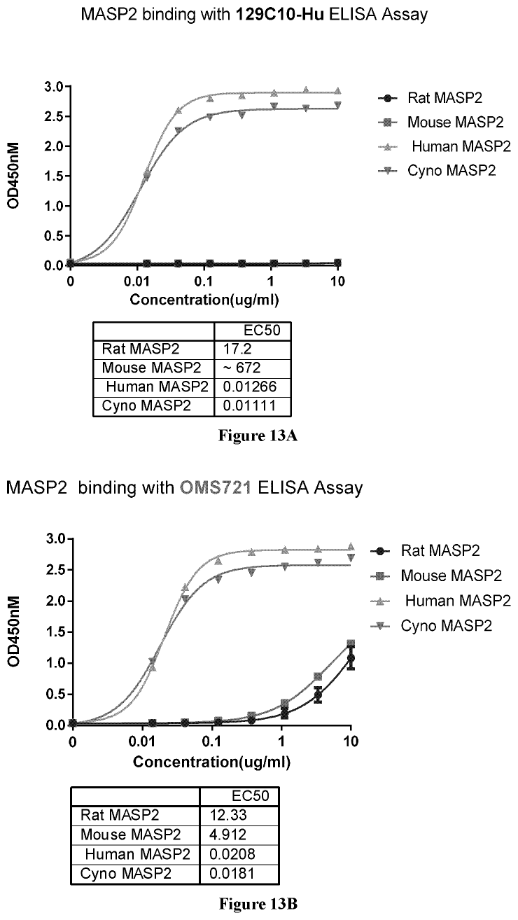

Rat/mouse MASP-2 were ordered from Cusbio company. Human/cyno MASP-2 were generated in house. ELISA assay was performed with following procedure. Coat MASP2 (1 μg/ml) of different species at 4° C. for overnight; wash three times with wash buffer; add blocking buffer (200 μL/well) at RT for 2h; wash three times; add serially diluted 129C10-hu or OMS721-analog as a control at RT for 1 h; wash three times; add mouse anti-human IgG4 Fc HRP (1:20000) at RT for 1 h; wash three times; detected with TMB for 2 min at OD450 nm. The EC50 of binding of 129C10-Hu and OMS721-analog to MASP-2 of different species were shown in FIG. 13A and FIG. 13B, respectively.

### Example 11: Cross-Reactivity of 129C10-Hu to Cynomolgus MASP-2 by Using C4 Activation Assay in Cynomolgus Serum

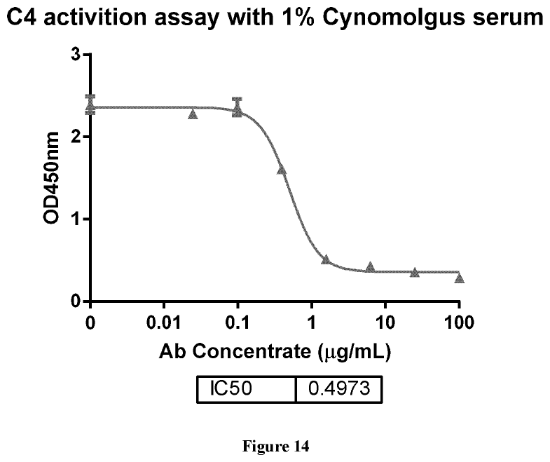

The assay method was same as described in Example 3 except for using cynomolgus serum. 100 μl per well 10 μg/ml mannan was coated onto ELISA plate at 4° C. for overnight. Antibodies were incubated with 1% cynomolgus serum for 45 min on ice. After washing and blocking the plate, antibody-serum mixture was added and then incubated at 37° C. for 90 min. After washing, the deposited activated C4 was detected with an HRP-linked anti-C4c antibody (Quidel-A211). As the result shown in FIG. 14, 129C10 blocked cynomolgus C4 activation with an IC₅₀ of 0.4973 μg/ml, suggesting 129C10 could bind with cynomolgus MASP-2 to block its activity.

### Example 12: The Selectivity of 129C10 in Blocking Activation of the MB-Lectin Complement Pathway

There are 3 pathways to initiate complement activation: classical pathway, MB-Lectin (MBL) pathway and alternative pathway. These pathways depend on different molecules for their initiation, while they converge to generate the same set of effector molecules like membrane attack complex (MAC). All three pathways are important parts of innate immunity and play different roles in defending different infections (Noris M, et. Al. 2013. JM.). Next, selectivity of blocking the MBL pathway by 129C10-hu was tested.

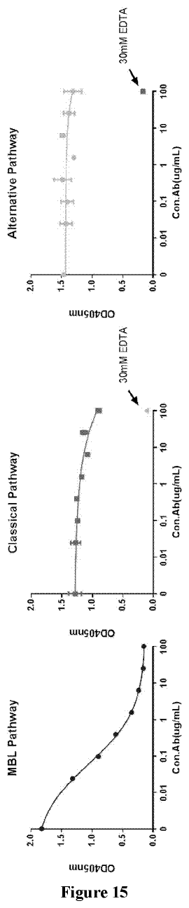

The Wieslab complement system screen kit (IBL America, Cat #COMPL 300 RUO) was used to determine the selectivity of the lead antibody 129C10-hu. The plate was pre-coated with mannan as an initiator for the MBL pathway, IgM as an initiator for classical pathway, and LPS as an initiator for alternative pathway. 129C10-hu was serially diluted with assay buffer containing human serum (Quidel, A113) and then incubated for 45 min on ice. Antibody-serum mixture was added to the plates and incubated at 37° C. for 60 min. After washing, the deposited MAC was detected with an AP-linked anti-C5b-9 antibody. EDTA was used as a positive control of blocking complement activation. As the results shown in FIG. 15, 129C10-hu only blocked complement activation initialized from the MBL pathway, but not the other two complement pathways, suggesting that 129C10-hu antibody selectively blocked the MBL pathway complement activation.

### Example 13: This Modification of 129C10-Hu to Extend Half-Life by Introducing YTE Mutation at Fc

Dall'Acqua W F et. al reported that introduction of triple mutation M252Y/S254T/T256E (YTE) into Fc portion of IgG could enhance its binding affinity to FcRn and pro-long its half-life in vivo (Dall'Acqua W F, et. al. 2006. JBC). Motavizumab-YTE, the first YTE mutation IgG in human was demonstrated well tolerated and exhibited an extended half-life in a phase I clinical trial (Robbie, G. J., et. al. 2013. AAC).

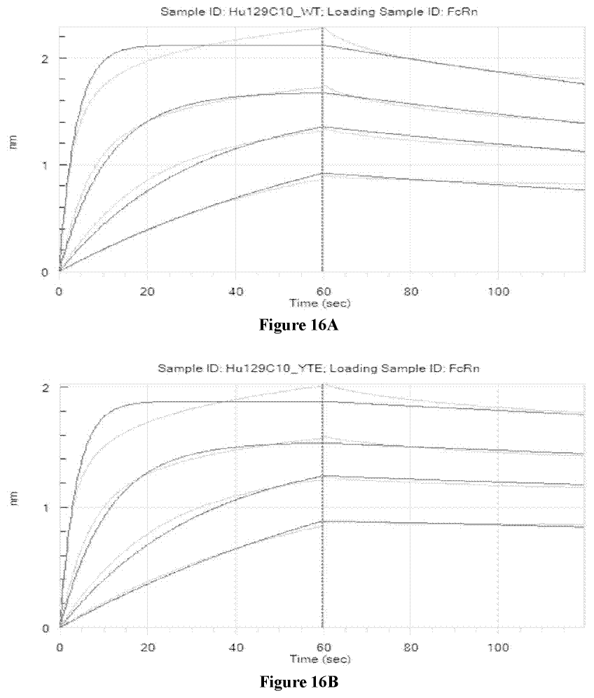

We introduced this M252Y/S254T/T256E (YTE) mutation into 129C10-hu to generate 129C10-hu-YTE. Its binding affinity to FcRn was evaluated with Bio-Layer Interferometry (ForteBio). 129C10-hu or 129C10-hu-YTE was diluted with ForteBio kinetics buffer (PBS pH 7.4, 0.1% BSA+0.002% Tween-20) to series concentrations of 500 nM, 167 nM, 56 nM, 19 nM and 0 nM. Human FcRn (FCGRT&B2M) protein with His tag was diluted with kinetics buffer to a concentration gradient of 100 nM. FcRn protein was immobilized onto Ni-NTA biosensors. The association and dissociation kinetics were recorded and analyzed. As the results shown in FIG. 16A and FIG. 16B and Table 5, the YTE mutation increased 129C10-hu binding affinity to human FcRn to 3 folds.

TABLE 5
129C10-hu and 129C10-hu-YTE binding affinity to human FcRn

| Sample ID | KD (M) | kon(1/Ms) | kdis(1/s) |
|---|---|---|---|
| 129C10-hu | 6.10E−09 | 5.20E+05 | 3.17E−03 |
| 129C10-hu-YTE | 1.88E−09 | 5.36E+05 | 1.01E−03 |

### Example 14: Pharmacokinetics (PK)/Pharmacodynamics (PD) Study of MASP-2 Antibody 129C10-Hu and 129C10-Hu-YTE in Cynomolgus

2 cynomolgus each group was administrated intravenously with 10 mg/kg 129C10-hu, 129C10-hu-YTE or OMS721-analog. Serum samples were collected at 0, 0.5, 2, 8, 24, 48, 72, 96, 168, 336, 504, 672, 840 hours post infusion. Antibody concentrations in serum and the potency of lectin pathway activation were tested.

Serum concentrations were determined by developed ELISA methods with detection range from 0.625-40 ng/mL. Microplate wells were pre-coated with a human IgG specific anti-IgG antibody [R10z8e6]. After blocking, standard (STD), quality control (QC) samples, matrix blank samples and the test samples were added. After washing, the biotin mouse anti-human IgG4 was added to the microplate wells and followed by Streptavidin labeled with HRP. TMB was added to the microplate wells. The conversion of OD values for QC and test samples into concentration was performed by comparison to a concurrently analyzed standard curve regressed according to a four-parameter logistic model.

For lectin pathway activation potency test, 10 μg/ml mannan was coated onto ELISA plate. Cynomolgus serum samples were diluted to 2% with C4 activation buffer. After washing and blocking of the plate, the diluted serum was added and incubated at 37° C. for 90 min. After washing, the deposited activated C4 was detected with an HRP-linked anti-C4c antibody (Quidel-A211).

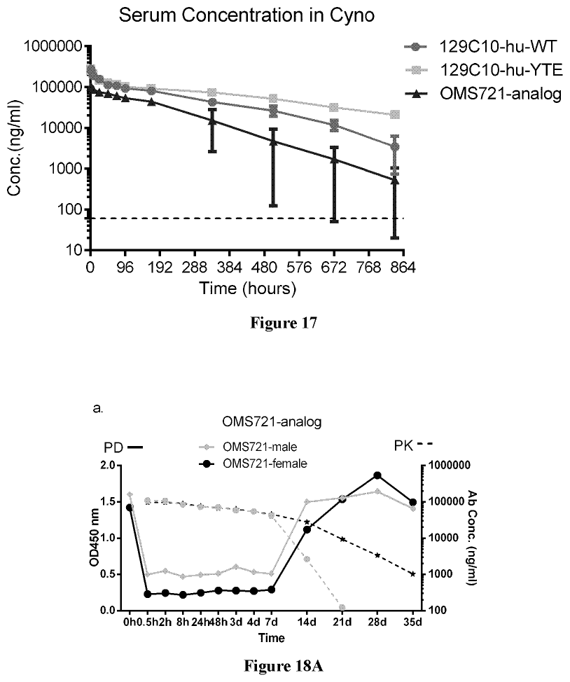

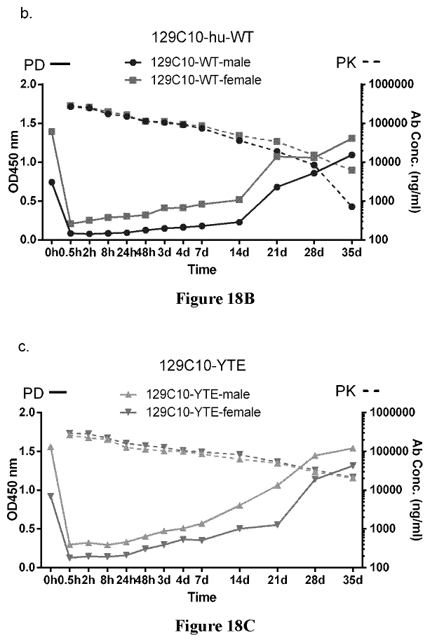

As the PK results shown in FIG. 17 and Table 6, half-life of 129C10-hu in cynomolgus is 164.77 hours longer than OMS721-analog (130.152 hours). YTE mutation increased the half-life of 129C10-hu to 274.404 hours. As the PD results shown in FIG. 18, the lectin pathway activation potency was inhibited to a basal level after 0.5h of antibody administration. The inhibition effect lasted for 2 weeks in OMS721-analog group, 3 weeks in 129C10-hu group and approximately 4 weeks in 129C10-hu-YTE group.

TABLE 6
Data summary of PK test in cynomolgus

| | | | 129C10- | OMS721- |
|---|---|---|---|---|
| PK | | 129C10-hu | hu-YTE | analog |
| Parameter | Unit | 10 mpk | 10 mpk | 10 mpk |
| t1/2 | h | 164.777 | 274.404 | 130.152 |
| Cmax | ng/ml | 277231.420 | 278092.525 | 102730.925 |
| AUC 0-t | ng/ml*h | 39897468.881 | 56133410.517 | 17892207.152 |
| Cl_obs | ml/day/kg | 5.894 | 3.725 | 13.270 |

In another separate study, male and female cynomolgus monkeys were assigned to five groups of 2 males and 2 females in each group, and dosed with 0 (vehicle control), 15, 20 and 295.8 mg/kg 129C10-hu via subcutaneous injection or 15 mg/kg 129C10-hu via intravenous injection at a volume of 3 mL/kg for 4 weeks (up to 5 doses). The vehicle control article used the drug Formulation Buffer.

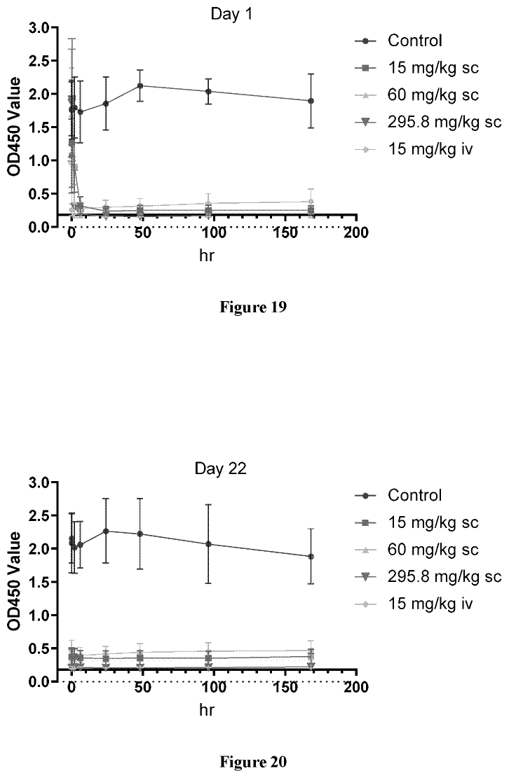

Blood samples were collected predose and at approximately 0.083, 2, 6, 24, 48, 96 and 168 hours post the first (see FIG. 19) and fourth doses (see FIG. 20), respectively. Complement 4c (C4c) in serum was analyzed with Enzyme-linked immune sorbent assay (ELISA). Briefly, Mannan (Sigma) was diluted to 10 μg/ml with coating buffer. 100 μl of mannan working solution was added to each well of 96 well plates. Incubated the plates overnight at 4° C. Washed the plates three times and then added 200 μl of blocking buffer and incubated for 1 h at room temperature. Dilute monkey serum to 2% with C4 activation buffer. Added 100 μl of diluted serum into 96 well plate and incubated at 37° C. for 70 min. Washed the plates three times with washing buffer. Added 100 μl of HRP linked anti-C4c antibody (diluted 1:5000 with block buffer) and incubated for 1 h at room temperature. Washed the plates three times with washing buffer. Added 100 μl TMB substrate solution into each well of 96 well plates, incubated at room temperature for 5-10 minutes. Added 50 μl stop solution into each well of plates. Read the plates at 450 nm. The optical density (OD) values at 450 nm (OD450) were expressed as individual values and the mean value at each time point of every animal was calculated.

Dose-dependent reduction of serum C4c was observed at ≥15 mg/kg following the first and fourth doses, and the reduction appeared from 2 hr post dose in animals dosed via S.C. and from 0.083 hr post dose in animals dosed via IV., the effect persisted in the whole dosing period with maximum effect at 24-96 hr post first dosing across the 3 dose levels by S.C. or IV., maximum mean reduction up to 88.4% in males and 92.8% in females were noted at 295.8 mg/kg by S.C. compared with baseline value. In conclusion, 129C10-hu exerted remarkably dose-dependent reduction effect on serum C4c in monkeys following S.C. or I.V. administration once weekly at 15, 60 or 295.8 mg/kg for 5 doses, suggesting serum C4c could be a potential pharmacodynamic marker.

What is claimed is:

1. An isolated antibody or an antigen binding fragment thereof that specifically binds to MASP-2, comprising:
a) a heavy chain CDR1 comprising the amino acid sequence of DYYIN (SEQ ID NO: 1),
a heavy chain CDR2 comprising the amino acid sequence of WIFPGSX₁SX₂YX₃X₄X₅X₆FX₇X₈ (SEQ ID NO: 2), and a heavy chain CDR3 comprising the amino acid sequence of GDRSGPFX₉Y (SEQ ID NO: 3); and/or
b) a light chain CDR1 comprising the amino acid sequence of KSSQSLLYSNGKTYLN (SEQ ID NO: 4), a light chain CDR2 comprising the amino acid sequence of LVSKLDS (SEQ ID NO: 5), and a light chain CDR3 comprising the amino acid sequence of VQX₁₀THFPFT (SEQ ID NO: 6);
wherein X₁ is E, D or G, X₂ is A or P, X₃ is H or Y, X₄ is S or N, X₈ is E or Q, X₆ is K or N, X₇ is K or Q, X₈ is A or G, X₉ is A or P, and X₁₀ is V or G.

2. The antibody or antigen binding fragment thereof of claim 1, comprising:
a) a heavy chain CDR1 comprising the amino acid sequence of SEQ ID NO: 1, and/or
b) a heavy chain CDR2 comprising the amino acid sequence selected from the group consisting of: SEQ ID NO: 7, SEQ ID NO: 8, SEQ ID NO: 9 and SEQ ID NO: 10, and/or
c) a heavy chain CDR3 comprising the amino acid sequence selected from the group consisting of: SEQ ID NO: 11 and SEQ ID NO: 12, and/or
d) a light chain CDR1 comprising the amino acid sequence of SEQ ID NO: 4, and/or
e) a light chain CDR2 comprising the amino acid sequence of SEQ ID NO: 5, and/or
f) a light chain CDR3 comprising the amino acid sequence selected from the group consisting of: SEQ ID NO: 13 and SEQ ID NO: 14.

3. An isolated antibody or an antigen binding fragment thereof that specifically binds to MASP-2, comprising:
a) a heavy chain CDR1 comprising the amino acid sequence of SEQ ID NO: 1, a heavy chain CDR2 comprising the amino acid sequence of SEQ ID NO: 7, and a heavy chain CDR3 comprising the amino acid sequence of SEQ ID NO: 11; or
b) a heavy chain CDR1 comprising the amino acid sequence of SEQ ID NO: 1, a heavy chain CDR2 comprising the amino acid sequence of SEQ ID NO: 9, and a heavy chain CDR3 comprising the amino acid sequence of SEQ ID NO: 12; or
c) a heavy chain CDR1 comprising the amino acid sequence of SEQ ID NO: 1, a heavy chain CDR2 comprising the amino acid sequence of SEQ ID NO: 10, and a heavy chain CDR3 comprising the amino acid sequence of SEQ ID NO: 11; or
d) a heavy chain CDR1 comprising the amino acid sequence of SEQ ID NO: 1, a heavy chain CDR2 comprising the amino acid sequence of SEQ ID NO: 8, and a heavy chain CDR3 comprising the amino acid sequence of SEQ ID NO: 11.

4. The antibody or antigen binding fragment thereof of claim 3, further comprising:
a) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
b) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 14.

5. The antibody or antigen binding fragment thereof of any one of the preceding claims, comprising:
a) a heavy chain CDR1 comprising the amino acid sequence of SEQ ID NO: 1, a heavy chain CDR2 comprising the amino acid sequence of SEQ ID NO: 7, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
b) a heavy chain CDR1 comprising the amino acid sequence of SEQ ID NO: 1, a heavy chain CDR2 comprising the amino acid sequence of SEQ ID NO: 9, and a heavy chain CDR3 comprising the amino acid sequence of SEQ ID NO: 12, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13; or
c) a heavy chain CDR1 comprising the amino acid sequence of SEQ ID NO: 1, a heavy chain CDR2 comprising the amino acid sequence of SEQ ID NO: 10, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 14; or
d) a heavy chain CDR1 comprising the amino acid sequence of SEQ ID NO: 1, a heavy chain CDR2 comprising the amino acid sequence of SEQ ID NO: 8, and a heavy chain CDR3 comprising the amino acid sequence of SEQ ID NO: 11, a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 13.

6. The antibody or antigen binding fragment thereof of any one of the preceding claims, comprising:
a) a heavy chain variable region comprising the amino acid sequence of SEQ ID NO: 15, or a sequence having at least 80% sequence identity thereof;
b) a heavy chain variable region comprising the amino acid sequence of SEQ ID NO: 17, or a sequence having at least 80% sequence identity thereof;
c) a heavy chain variable region comprising the amino acid sequence of SEQ ID NO: 18, or a sequence having at least 80% sequence identity thereof;
d) a heavy chain variable region comprising the amino acid sequence of SEQ ID NO: 20, or a sequence having at least 80% sequence identity thereof;
e) a heavy chain variable region comprising the amino acid sequence of SEQ ID NO: 22, or a sequence having at least 80% sequence identity thereof;
f) a heavy chain variable region comprising the amino acid sequence of SEQ ID NO: 24, or a sequence having at least 80% sequence identity thereof; or
g) a heavy chain variable region comprising the amino acid sequence of SEQ ID NO: 26, or a sequence having at least 80% sequence identity thereof.

7. The antibody or antigen binding fragment thereof of any one of the preceding claims, comprising:
a) a light chain variable region comprising the amino acid sequence of SEQ ID NO: 16, or a sequence having at least 80% sequence identity thereof;
b) a light chain variable region comprising the amino acid sequence of SEQ ID NO: 19, or a sequence having at least 80% sequence identity thereof;
c) a light chain variable region comprising the amino acid sequence of SEQ ID NO: 28, or a sequence having at least 80% sequence identity thereof; or
d) a light chain variable region comprising the amino acid sequence of SEQ ID NO: 30, or a sequence having at least 80% sequence identity thereof.

8. The antibody or antigen binding fragment thereof of any one of the preceding claims, wherein the heavy chain variable region comprises an amino acid sequence having at least 80% sequence identity to SEQ ID NO: 15, SEQ ID NO: 17, SEQ ID NO: 18, SEQ ID NO: 20, SEQ ID NO: 22, SEQ ID NO: 24, or SEQ ID NO: 26, and/or the light chain variable region comprises an amino acid sequence having at least 80% sequence identity to SEQ ID NO: 16, SEQ ID NO: 19, SEQ ID NO: 28, or SEQ ID NO: 30.

9. The antibody or antigen binding fragment thereof of any one of the preceding claims, comprising:
a) a heavy chain variable region comprising the amino acid sequence of SEQ ID NO: 15, and a light chain variable region comprising the amino acid sequence of SEQ ID NO: 16;
b) a heavy chain variable region comprising the amino acid sequence of SEQ ID NO: 17, and a light chain variable region comprising the amino acid sequence of SEQ ID NO: 16;
c) a heavy chain variable region comprising the amino acid sequence of SEQ ID NO: 18, and a light chain variable region comprising the amino acid sequence of SEQ ID NO: 19;
d) a heavy chain variable region comprising the amino acid sequence of SEQ ID NO: 20, and a light chain variable region comprising the amino acid sequence of SEQ ID NO: 28;
e) a heavy chain variable region comprising the amino acid sequence of SEQ ID NO: 20, and a light chain variable region comprising the amino acid sequence of SEQ ID NO: 30;
f) a heavy chain variable region comprising the amino acid sequence of SEQ ID NO: 22, and a light chain variable region comprising the amino acid sequence of SEQ ID NO: 28;
g) a heavy chain variable region comprising the amino acid sequence of SEQ ID NO: 22, and a light chain variable region comprising the amino acid sequence of SEQ ID NO: 30;
h) a heavy chain variable region comprising the amino acid sequence of SEQ ID NO: 24, and a light chain variable region comprising the amino acid sequence of SEQ ID NO: 28;
i) a heavy chain variable region comprising the amino acid sequence of SEQ ID NO: 24, and a light chain variable region comprising the amino acid sequence of SEQ ID NO: 30;
j) a heavy chain variable region comprising the amino acid sequence of SEQ ID NO: 26, and a light chain variable region comprising the amino acid sequence of SEQ ID NO: 28; or
k) a heavy chain variable region comprising the amino acid sequence of SEQ ID NO: 26, and a light chain variable region comprising the amino acid sequence of SEQ ID NO: 30.

10. The antibody or antigen binding fragment thereof of any one of the preceding claims, further comprising one or more amino acid residue mutations yet retaining binding specificity to human MASP-2.

11. The antibody or antigen binding fragment thereof of claim 10, wherein at least one of the mutations is conservative substitution, or all of the mutations are conservative substitutions.

12. The antibody or antigen binding fragment thereof of claim 10 or 11, wherein at least one of the mutations is in one or more of the CDR sequences, and/or in one or more of the non-CDR sequences of the heavy chain variable region or light chain variable region.

13. The antibody or antigen binding fragment thereof of any one of the preceding claims, further comprising an immunoglobulin constant region, optionally comprising a heavy chain constant region of IgG, and/or a light chain constant region.

14. The antibody or antigen binding fragment thereof of claim 13, wherein the constant region comprises a mouse constant region, a rabbit constant region, or a human constant region, optionally wherein the constant region comprises a constant region of human IgG1, IgG2, IgG3, or IgG4.

15. The antibody or antigen binding fragment thereof of claim 13, wherein the heavy chain constant region comprises one or more amino acid substitutions relative to a wild-type human IgG constant region at amino acid residue 252, 254 or 256,
optionally the amino acid substitution at amino acid residue 252 is a substitution with tyrosine, the amino acid substitution at amino acid residue 254 is a substitution with threonine, an amino acid substitution at amino acid residue 256 is a substitution with glutamic acid.

16. The antibody or antigen binding fragment thereof of claim 15, wherein the heavy chain constant region comprises a sequence having at least 80% sequence identity thereof, provided that the amino acid residue 252 is substituted with tyrosine, the amino acid residue 254 is substituted with threonine, and the amino acid residue 256 is substituted with glutamic acid.

17. The antibody or antigen binding fragment thereof of any one of the preceding claims, which is a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g. glycosylated antibody).

18. The antibody or antigen binding fragment thereof of any one of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

19. The antibody or antigen binding fragment thereof of any one of the preceding claims, which specifically binds to MASP-2 and has no detectable cross-reactivity with C1s, C1r, MASP1 or MASP3.

20. The antibody or antigen binding fragment thereof of any one of the preceding claims linked to one or more conjugate moieties.

21. The antibody or antigen binding fragment thereof of claim 20, wherein the conjugate moiety comprises a clearance-modifying agent, a chemotherapeutic agent, a toxin, a radioactive isotope, a lanthanide, a luminescent label, a fluorescent label, an enzyme-substrate label, or a therapeutic agent.

22. A monoclonal antibody or an antigen binding fragment thereof, which competes for binding to MASP-2 with the antibody or antigen binding fragment thereof of any one of the preceding claims.

23. A pharmaceutical composition comprising the antibody or antigen binding fragment thereof of any one of the preceding claims, and a pharmaceutically acceptable carrier.

24. An isolated polynucleotide encoding the antibody or antigen binding fragment thereof of any one of the preceding claims.

25. A vector comprising the isolated polynucleotide of claim 24.

26. A host cell comprising the vector of claim 25.

27. A method of expressing the antibody or antigen binding fragment thereof of any one of claims 1-22, comprising culturing the host cell of claim 26 under the condition at which the polynucleotide of claim 24 is expressed.

28. A method of inhibiting MASP-2 dependent complement activation in a subject in need thereof, comprising administering to the subject a therapeutically effective amount of the antibody or antigen binding fragment thereof of any one of claims 1-22 or the pharmaceutical composition of claim 23, thereby inhibiting MASP-2 dependent complement activation in the subject.

29. A method of treating a disease or condition in a subject that would benefit from inhibition of MASP-2 dependent complement activation, comprising administering to the subject a therapeutically effective amount of the antibody or antigen binding fragment thereof of any one of claims 1-22 or the pharmaceutical composition of claim 23.

30. A method of reducing level of serum C4 in a subject, comprising administering to the subject a therapeutically effective amount of the antibody or antigen binding fragment thereof of any one of claims 1-22 or the pharmaceutical composition of claim 23, thereby reducing the level of serum C4 in the subject.

31. A method of treating a disease or condition in a subject that would benefit from reduction of serum C4 level, or treating or preventing a condition or a disease associated with abnormal serum C4 level, comprising administering to the subject a therapeutically effective amount of the antibody or antigen binding fragment thereof of any one of claims 1-22 or the pharmaceutical composition of claim 23.

32. The method of claim 29 or 31, wherein the disease or condition is an autoimmune disease, a vascular condition, an ischemia-reperfusion injury, atherosclerosis, an inflammation, a pulmonary condition, extracorporeal reperfusion procedure, a musculoskeletal condition, a renal condition, a skin condition, an organ or tissue transplant procedure, a nervous system disorder or injury, a blood disorder, urogenital condition, a nonobese diabetes or a complication associated with Type 1 or Type 2 diabetes, cancer, endocrine disorder, or an ophthalmologic condition.

33. The method of claim 32, wherein the autoimmune disease comprises thrombotic microangiopathies (TMAs), atypical hemolytic uremic syndrome (aHUS), hematopoietic transplant-associated thrombotic microangiopathy (TA-TMA), lupus nephritis, systemic lupus erythematosus (SLE) and IgA nephropathy,
the vascular condition comprises a cardiovascular condition, a cerebrovascular condition, a peripheral (e.g., musculoskeletal) vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy and percutaneous transluminal coronary angioplasty (PTCA), the ischemia-reperfusion injury comprises an ischemia-reperfusion injury associated with aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures,
the inflammation comprises inflammatory gastrointestinal disorder comprising pancreatitis, Crohn's disease, ulcerative colitis, irritable bowel syndrome and diverticulitis, the pulmonary condition comprises acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to burn, non-cardiogenic pulmonary edema, transfusion-related respiratory depression, emphysema, cystic fibrosis, SARS-CoV, MERS-CoV and SARS-CoV-2 (Covid-19) related condition,
the extracorporeal reperfusion procedure comprises hemodialysis, plasmapheresis, leukapheresis, extracorporeal membrane oxygenator (ECMO), heparin-induced extracorporeal membrane oxygenation LDL precipitation (HELP) and cardiopulmonary bypass (CPB),
the musculoskeletal condition comprises osteoarthritis, rheumatoid arthritis, juvenile rheumatoid arthritis, gout, neuropathic arthropathy, psoriatic arthritis, spondyloarthropathy, crystalline arthropathy and systemic lupus erythematosus (SLE), the renal condition comprises mesangioproliferative glomerulonephritis, membranous glomerulonephritis, membranoproliferative glomerulonephritis (mesangiocapillary glomerulonephritis), acute postinfectious glomerulonephritis (poststreptococcal glomerulonephritis), cryoglobulinemic glomerulonephritis, lupus nephritis, Henoch-Schonlein purpura nephritis and IgA nephropathy,
the skin condition comprises psoriasis, autoimmune bullous dermatoses, eosinophilic spongiosis, bullous pemphigoid, Epidermolysis bullosa acquisita (EBA), herpes gestationis, thermal burn injury and chemical burn injury,
the organ or tissue transplant procedure comprises organ allotransplantation, organ xenotransplantation organ and tissue graft,
the nervous system disorder or injury comprises multiple sclerosis, myasthenia gravis, Huntington's disease, amyotrophic lateral sclerosis, Guillain Barre syndrome, reperfusion following stroke, degenerative discs, cerebral trauma, Parkinson's disease, Alzheimer's disease, Miller-Fisher syndrome, cerebral trauma and/or hemorrhage, demyelination and meningitis,
the blood disorder comprises sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome,
the urogenital condition comprises painful bladder disease, sensory bladder disease, chronic abacterial cystitis, interstitial cystitis, infertility, placental dysfunction and miscarriage and pre-eclampsia,
the endocrine disorder comprises Hashimoto's thyroiditis, stress, anxiety and hormonal disorders involving regulated release of prolactin, growth or other insulin-like growth factor and adrenocorticotropin from the pituitary,
the ophthalmologic condition comprises age-related macular degeneration.

34. The method of any one of claims 28-33, further comprising administration of a second therapeutic agent.

35. The method of any one of claims 28-34, wherein the subject is human.

36. The method of any one of claims 28-35, wherein the administration is via oral, nasal, intravenous, subcutaneous, sublingual, or intramuscular administration.

37. Use of the antibody or antigen binding fragment thereof of any one of claims 1-22 in the manufacture of a medicament for treating a MASP-2 dependent complement activation related disease or condition in a subject.

38. A kit comprising the antibody or antigen binding fragment thereof of any one of claims 1-22.


SEQUENCE LISTING (43 sequences)

SEQ ID NO: 1 - protein, 5 aa, Artificial Sequence
  Synthetic
DYYIN

SEQ ID NO: 2 - protein, 17 aa, Artificial Sequence
  Synthetic
  misc_feature (7)..(7): Xaa can be Glu, Asp or Gly.
  misc_feature (9)..(9): Xaa can be Ala or Pro.
  misc_feature (11)..(14): Xaa at position 11 can be His or Tyr, Xaa at position 12 can be Ser or Asn, Xaa at position 13 can be Glu or Gln, and Xaa at position 14 can be Lys or Asn.
  misc_feature (16)..(17): Xaa at position 16 can be Lys or Gln, and Xaa at position 17 can be Ala or Gly.
WIFPGSXSXYXXXXFXX

SEQ ID NO: 3 - protein, 9 aa, Artificial Sequence
  Synthetic
  misc_feature (8)..(8): Xaa can be Ala or Pro.
GDRSGPFXY

SEQ ID NO: 4 - protein, 16 aa, Artificial Sequence
  Synthetic
KSSQSLLYSNGKTYLN

SEQ ID NO: 5 - protein, 7 aa, Artificial Sequence
  Synthetic
LVSKLDS

SEQ ID NO: 6 - protein, 9 aa, Artificial Sequence
  Synthetic
  misc_feature (3)..(3): Xaa can be Val or Gly.
VQXTHFPFT

SEQ ID NO: 7 - protein, 17 aa, Artificial Sequence
  Synthetic
WIFPGSESAYHSEKFKA

SEQ ID NO: 8 - protein, 17 aa, Artificial Sequence
  Synthetic
WIFPGSESAYHSQKFQG

SEQ ID NO: 9 - protein, 17 aa, Artificial Sequence
  Synthetic
WIFPGSDSAYYNEKFKG

SEQ ID NO: 10 - protein, 17 aa, Artificial Sequence
  Synthetic
WIFPGSGSPYYNENFKG

SEQ ID NO: 11 - protein, 9 aa, Artificial Sequence
  Synthetic
GDRSGPFAY

SEQ ID NO: 12 - protein, 9 aa, Artificial Sequence
  Synthetic
GDRSGPFPY

SEQ ID NO: 13 - protein, 9 aa, Artificial Sequence
  Synthetic
VQVTHFPFT

SEQ ID NO: 14 - protein, 9 aa, Artificial Sequence
  Synthetic
VQGTHFPFT

SEQ ID NO: 15 - protein, 118 aa, Artificial Sequence
  Synthetic
QVQLQQSGPELEKPGTSVKISCEASGYTFTDYYINWVQQRPGQGLEWIGWIFPGSESAYHSEKFKAKATLTVDTSSTTAYMLLTSLTSEDSAVYFCTRGDRSGPFAYWGQGTLVTVSA

SEQ ID NO: 16 - protein, 112 aa, Artificial Sequence
  Synthetic
DVVMTQTPLTLSVTIGQPASISCKSSQSLLYSNGKTYLNWLLQRPGQSPKRLIYLVSKLDSGVPDRFTGSGSGADFTLKISRVEAEDLGVYYCVQVTHFPFTFGTGTKLEIK

SEQ ID NO: 17 - protein, 118 aa, Artificial Sequence
  Synthetic
QVQLQQSGPELVKPGASVKISCKASGYTFTDYYINWVKQRPGQGLEWVGWIFPGSDSAYYNEKFKGKATLTVDTSSSTAYMLLSSLTSEDSAVYFCARGDRSGPFPYWGQGTLVTVSA

SEQ ID NO: 18 - protein, 118 aa, Artificial Sequence
  Synthetic
QVQLQQSGPDLVKPGTSVKISCTASGYTFTDYYINWVKQRPGQGLEWIGWIFPGSGSPYYNENFKGRAMFTVDYSSSSAYMLLSSLTSEDSAVYFCTRGDRSGPFAYWGQGTLVTVSA

SEQ ID NO: 19 - protein, 112 aa, Artificial Sequence
  Synthetic
DVVMTQTPLTLSVTIGQPASISCKSSQSLLYSNGKTYLNWLLQRPGQSPKRLIYLVSKLDSGVPDRFTGSGSGADFTLKISRVEAEDLGVYYCVQGTHFPFTFGTGTKLEIK

SEQ ID NO: 20 - protein, 118 aa, Artificial Sequence
  Synthetic
QVQLVQSGAEVKKPGASVKVSCKASGYTFTDYYINWVRQAPGQGLEWMGWIFPGSESAYHSEKFKARVTMTVDTSISTAYMELSRLRSDDTAVYYCTRGDRSGPFAYWGQGTLVTVSS

SEQ ID NO: 21 - skipped

SEQ ID NO: 22 - protein, 118 aa, Artificial Sequence
  Synthetic
QVQLVQSGAEVKKPGASVKVSCKASGYTFTDYYINWVRQAPGQGLEWMGWIFPGSESAYHSEKFKARATLTVDTSISTAYMELSRLRSDDTAVYYCTRGDRSGPFAYWGQGTLVTVSS

SEQ ID NO: 23 - skipped

SEQ ID NO: 24 - protein, 118 aa, Artificial Sequence
  Synthetic
QVQLVQSGAEVKKPGASVKVSCKASGYTFTDYYINWVRQAPGQGLEWMGWIFPGSESAYHSQKFQGRVTMTVDTSISTAYMELSRLRSDDTAVYYCTRGDRSGPFAYWGQGTLVTVSS

SEQ ID NO: 25 - skipped

SEQ ID NO: 26 - protein, 118 aa, Artificial Sequence
  Synthetic
QVQLVQSGAEVKKPGASVKVSCKASGYTFTDYYINWVRQAPGQGLEWMGWIFPGSESAYHSQKFQGRATLTVDTSISTAYMELSRLRSDDTAVYYCTRGDRSGPFAYWGQGTLVTVSS

SEQ ID NO: 27 - skipped

SEQ ID NO: 28 - protein, 112 aa, Artificial Sequence
  Synthetic
DVVMTQSPLSLPVTLGQPASISCKSSQSLLYSNGKTYLNWLQQRPGQSPRRLIYLVSKLDSGVPDRFSGSGSGTDFTLKISRVEAEDVGVYYCVQVTHFPFTFGQGTKLEIK

SEQ ID NO: 29 - protein, 219 aa, Artificial Sequence
  Synthetic
DVVMTQSPLSLPVTLGQPASISCKSSQSLLYSNGKTYLNWLQQRPGQSPRRLIYLVSKLDSGVPDRFSGSGSGTDFTLKISRVEAEDVGVYYCVQVTHFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 112 aa, Artificial Sequence
  Synthetic
DVVMTQSPLSLPVTLGQPASISCKSSQSLLYSNGKTYLNWLQQRPGQSPRRLIYLVSKLDSGVPDRFSGSGSGADFTLKISRVEAEDVGVYYCVQVTHFPFTFGQGTKLEIK

SEQ ID NO: 31 - skipped

SEQ ID NO: 32 - protein, 445 aa, Artificial Sequence
  Synthetic
QVTLKESGPVLVKPTETLTLTCTVSGFSLSRGKMGVSWIRQPPGKALEWLAHIFSSDEKSYRTSLKSRLTISKDTSKNQVVLTMTNMDPVDTATYYCARIRRGGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 213 aa, Artificial Sequence
  Synthetic
QPVLTQPPSLSVSPGQTASITCSGEKLGDKYAYWYQQKPGQSPVLVMYQDKQRPSGIPERFSGSNSGNTATLTISGTQAMDEADYYCQAWDSSTAVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 107 aa, Homo sapiens [sequence omitted]

SEQ ID NO: 35 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 445 aa, Artificial Sequence
  Synthetic
QVQLVQSGAEVKKPGASVKVSCKASGYTFTDYYINWVRQAPGQGLEWMGWIFPGSESAYHSEKFKARVTMTVDTSISTAYMELSRLRSDDTAVYYCTRGDRSGPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 38 - protein, 100 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 39 - protein, 686 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 40 - protein, 685 aa, Mus musculus
MRLLIFLGLLWSLVATLLGSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSALCSGQVFTGRSGYLSSPEYPQPYPKLSSCTYSIRLEDGFSVILDFVESFDVETHPEAQCPYDSLKIQTDKGEHGPFCGKTLPPRIETDSHKVTITFATDESGNHTGWKIHYTSTARPCPDPTAPPNGSISPVQAIYVLKDRFSVFCKTGFELLQGSVPLKSFTAVCQKDGSWDRPMPECSIIDCGPPDDLPNGHVDYITGPEVTTYKAVIQYSCEETFYTMSSNGKYVCEADGFWTSSKGEKLPPVCEPVCGLSTHTIGGRIVGGQPAKPGDFPWQVLLLGQTTAAAGALIHDNWVLTAAHAVYEKRMAASSLNIRMGILKRLSPHYTQAWPEEIFIHEGYTHGAGFDNDIALIKLKNKVTINGSIMPVCLPRKEAASLMRTDFTGTVAGWGLTQKGLLARNLMFVDIPIADHQKCTAVYEKLYPGVRVSANMLCAGLETGGKDSCRGDSGGALVFLDNETQRWFVGGIVSWGSINCGAADQYGVYTKVINYIPWIENIISNF

SEQ ID NO: 41 - protein, 20 aa, Homo sapiens
MYRMQLLSCIALSLALVTNS

SEQ ID NO: 42 - protein, 695 aa, Artificial Sequence
  Synthetic
MYRMQLLSCIALSLALVTNSSKWPEPVFGRLVSPGFPEKYADHQDRSWTLTAPPGYRLRLYFTHFDLELSYRCEYDFVKLSSGTKVLATLCGQESTDTEQAPGNDTFYSLGPSLKVTFHSDYSNEKPFTGFEAFYAAEDVDECRVSLGDSVPCDHYCHNYLGGYYCSCRAGYVLHQNKHTCSALCSGQVFTGRSGYLSSPEYPQPYPKLSSCTYSIRLEDGFSVILDFVESFDVETHPEAQCPYDSLKIQTDKGEHGPFCGKTLPPRIETDSHKVTITFATDESGNHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDFDYKDDDDK

SEQ ID NO: 43 - protein, 686 aa, Macaca fascicularis
MRLLTLLGLLCGSVATPLGPKWPEPVFGRLASPGFPGEYANDQERRWTLTAPPGYRLRLYFTHFDLELSHLCEYDFVKLSSGAKVLATLCGHESTDTERAPGNDTFYSLGSSLDITFRSDYSNEKPFTGFEAFYAAEDIDECQVAPGEAPACDHHCHNHLGGFYCSCRVGYILHRNKRTCSALCSGQVFTQRSGELSSPEYPQPYPKLSSCTYSIRLEEGFSVILDFVESFDVETHPETLCPYDFLKIQIDSEEHGPFCGKTLPRRIETKSNTVTITFVTDESGDHTGWKIHYTSTAQPCPYPMAPPNGHLSPVQAKYILKDSFSIFCEPGYELLQGHLPLKSFAAVCQKDGSWDQPMPSCSIVDCGPPDDLPSGRVEYITGPEVTTYKAVIQYSCEETFYTMKVNDGKYVCEADGFWTSSKGERSPPVCEPVCGLSARTTGGRIYGGQKAKPGDFPWQVLILGGSTAAGALLYDNWVLTAAHAIYEQKHDASSLDIRLGALKRLSPHYTQAWAEAVFIHEGYTHDAGFDNDIALIKLNNKVVINSNITPICLPRKEAESFMRTDDIGTASGWGLTQRGLLARNLMYVDIPIVDHQKCTAAYEKPPYSGGSVTANMLCAGLESGGKDSCRGDSGGALVFLDNETQRWFVGGIVSWGSMNCGEAGQYGVYTKVINYIPWIKNIISNF